(19) United States
(12) Patent Application Publication
(10) Pub. No.: US 2002/0192230 A1
(43) Pub. Date: Dec. 19, 2002

(54) Therapeutic formulations using heat shock/stress protein-peptide complexes

(75) Inventor: Pramod K. Srivastava, Avon, CT (US)

Correspondence Address: PENNIE AND EDMONDS, 1155 AVENUE OF THE AMERICAS, NEW YORK, NY, 100362711

(73) Assignee: University of Connecticut Health Center

(21) Appl. No.: 10/126,368

(22) Filed: Apr. 19, 2002

Related U.S. Application Data
(63) Continuation of application No. PCT/US01/28840, filed on Sep. 17, 2001.
(60) Provisional application No. 60/232,779, filed on Sep. 15, 2000.

(51) Int. Cl. A61K 39/00

(52) U.S. Cl. 424/185.1; 424/277.1

(57) ABSTRACT

The present invention relates to methods for making compositions comprising heat shock proteins or alpha (2) macroglobulin (“α2M”), which compositions are immunogenic against a type of cancer or an agent of an infectious disease, and the compositions produced by the methods described herein. The invention further relates to methods for eliciting an immune response and the prevention and treatment of primary and metastatic neoplastic diseases and infectious diseases. Specifically, the present invention provides a method of eliciting an immune response comprise administering to an individual a composition made by mixing an amount of a purified first complex comprising a first heat shock protein or α2M complexed to a peptide which displays antigenicity of an antigen of said type of cancer or antigenicity of an antigen of an agent of said infectious disease; and an equal or greater amount of a second heat shock protein or α2M that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or cells infected with said agent of infectious disease, respectively. Optionally, the methods further comprise administering antigen presenting cells sensitized with hsp-peptide or α2M-peptide complexes comprising peptides antigenic to cancer cells or to an agent of an infectious disease.

This application claims the benefit under 35 U.S.C. § 119(e) of U.S. Provisional Application No. 60/232,779 filed Sep. 15, 2000, which is incorporated by reference herein in its entirety.

This invention was made with government support under grant numbers CA44786 and CA64394 awarded by the National Institutes of Health. The government has certain rights in the invention.

### INTRODUCTION

The present invention relates to methods for preparing compositions that are useful for the prevention and treatment of infectious diseases, and primary and metastatic neoplastic diseases, and the compositions prepared by these methods. The compositions comprise a first complex which comprises a heat shock protein (hsp) or α-2-macrogobulin (“α2M”) complexed to a peptide that displays antigenicity of an antigen of an agent of an infectious disease or a type of cancer (the Specific Antigen, said complex being the Specific Complex), and a second hsp or α2M optionally complexed to a peptide which peptide is not a specific antigen (a non-specific antigen). The second hsp or α2M, whether complexed to a peptide or not, acts as a diluent (the Diluent). The composition comprising a Specific Complex and a Diluent is referred to as a Diluted Complex. The ratio of the Specific Complex to the Diluent in a Diluted Complex is at least 1:1. The hsps include but are not limited to hsp70, hsp90, gp96, calreticulin, hsp 110, or grp170, alone or in combination with each other, noncovalently or covalently bound to antigenic molecules. In the practice of the invention, Diluted Complexes may be administered alone or in combination with the administration of antigen presenting cells sensitized with a Specific Complex.

### BACKGROUND OF THE INVENTION

2.1. Vaccines

Vaccination has eradicated certain diseases such as polio, tetanus, chicken pox, and measles in many countries. This approach has exploited the ability of the immune system to resist and prevent infectious diseases.

Traditional ways of preparing vaccines include the use of inactivated or attenuated pathogens. A suitable inactivation of the pathogenic microorganism renders it harmless as a biological agent but does not destroy its immunogenicity. Injection of these “killed” particles into a host will then elicit an immune response capable of preventing a future infection with a live microorganism. However, a major concern in the use of inactivated pathogens as vaccines is the failure to inactivate all the microorganisms. Even when this is accomplished, since killed pathogens do not multiply in their host, or for other unknown reasons, the immunity achieved is often incomplete, short lived and requires multiple immunizations. Finally, the inactivation process may alter the microorganism's antigens, rendering them less effective as immunogens.

Attenuation refers to the production of strains of pathogenic microorganisms which have essentially lost their disease-producing ability. One way to accomplish this is to subject the microorganism to unusual growth conditions and/or frequent passage in cell culture. Mutants are then selected which have lost virulence but yet are capable of eliciting an immune response. Attenuated pathogens often make good immunogens as they actually replicate in the host cell and elicit long lasting immunity. However, several problems are encountered with the use of live vaccines, the most worrisome being insufficient attenuation and the risk of reversion to virulence.

An alternative to the above methods is the use of subunit vaccines. This involves immunization only with those components which contain the relevant immunological material. A new promising alternative is the use of DNA or RNA as vaccines. Such genetic vaccines have progressed from an idea to entities being studied in clinical trials (See, Weiner and Kennedy, July 1999, Scientific American, pp. 50-57).

Vaccines are often formulated and inoculated with various adjuvants. The adjuvants aid in attaining a more durable and higher level of immunity using small amounts of antigen or fewer doses than if the immunogen were administered alone. However, the mechanism of adjuvant action is unpredictable, complex and not completely understood (See Suzue et al., 1996, Basel: Birkhauser Verlag, 454-55).

Because of the risks associated with inactivated and attenuated pathogens, the ability to boost or amplify an immune response with minimal quantities of a vaccine would be ideal and advantageous. Furthermore, as the mechanism of adjuvants is not completely understood and is still unpredictable, alternative methods of boosting a subject's immune response with current methods of vaccination is highly desirable.

2.2. Heat Shock Proteins and their Roles in Antigen Presentation

Heat shock proteins (hsps), also known as stress proteins, are intracellular molecules that are abundant, soluble, and highly conserved. As intracellular chaperones, hsps participate in many biochemical pathways of protein maturation, and function actively, during times of stress and normal cellular homeostasis (See Mizzen, 1998, Biotherapy 10:174). Many stresses can disrupt the three-dimensional structure, or folding, of a cell's proteins. Left uncorrected, mis-folded proteins form aggregates that may eventually kill the cell. Hsps bind to those damaged proteins, helping them refold into their proper conformations. In normal (unstressed) cellular homeostasis, hsps are required for cellular metabolism. Hsps help newly synthesized polypeptides fold and thus prevent premature interactions with other proteins. Also, hsps aid in the transport of proteins throughout the cell's various compartments.

The major hsps can accumulate to very high levels in stressed cells, but they occur at low to moderate levels in cells that have not been stressed. For example, the highly inducible mammalian hsp70 is hardly detectable at normal temperatures but becomes one of the most actively synthesized proteins in the cell upon heat shock (Welch et al., 1985, J. Cell. Biol. 101:1198-1211). In contrast, hsp90and hsp60 proteins are abundant at normal temperatures in most, but not all, mammalian cells and are further induced by heat (Lai et al., 1984, Mol. Cell. Biol. 4:2802-2810; van Bergen en Henegouwen et al., 1987, Genes Dev. 1:525-531).

Hsps have been found to have immunological and antigenic properties. Immunization of mice with gp96 or p84/86 isolated from a particular tumor rendered the mice immune to that particular tumor, but not to antigenically distinct tumors. (Srivastava, et al., 1988, Immunogenetics 28:205-207; Srivastava et al., 1991, Curr. Top. Microbiol. Immunol. 167:109-123). Further, hsp70 was shown to elicit immunity to the tumor from which it was isolated but not to antigenically distinct tumors. However, hsp70 depleted of peptides was found to lose its immunogenic activity (Udono and Srivastava, 1993, J. Exp. Med. 178:1391-1396). These observations suggested that the heat shock proteins are not immunogenic per se, but form noncovalent complexes with antigenic peptides, and the complexes can elicit specific immunity against the antigenic peptides (Srivastava, 1993, Adv. Cancer Res. 62:153-177; Udono et al., 1994, J. Immunol., 152:5398-5403; Suto et al., 1995, Science, 269:1585-1588).

Based on the observations by Srivastava and others about the immunogenicity of tumors and more specifically of heat shock/stress protein preparations derived from tumors, methods were developed for the isolation of stress protein-peptide complexes from mammalian tumor cells and administering the complexes back to the mammals (U.S. Pat. No. 5,750,119). Stress protein complexes derived from tumors were able to confer resistance to challenges with cells obtained from the same tumors (U.S. Pat. No. 5,837,251). Stress protein preparations from carcinoma cells of higher immunogenicity provided greater resistance than did stress protein preparations from carcinoma cells of lower immunogenicity against their respective tumor cell types (U.S. Pat. No. 5,837,251). See also U.S. Pat. Nos. 6,017,540 and 5,830,464 to Srivastava.

The use of hsp-peptide complexes for sensitizing antigen presenting cells in vitro for use in adoptive immunotherapy is described in U.S. Pat. No. 5,985,270.

Hsp-peptide complexes can also be isolated from pathogen-infected cells and used for the treatment and prevention of infection caused by the pathogen, such as viruses, and other intracellular pathogens, including bacteria, protozoa, fungi and parasites; see U.S. Pat. No. 5,961,979.

Immunogenic hsp-peptide complexes can also be prepared by in vitro complexing of hsps and antigenic peptides, and the uses of such complexes for the treatment and prevention of cancer and infectious diseases has been described in U.S. Pat. Nos. 5,935,576 and 6,030,618. The use of heat shock protein in combination with a defined antigen for the treatment of cancer and infectious diseases have also been described in PCT publication WO97/06821 dated Feb. 27, 1997.

The purification of hsp-peptide complexes from cell lysate has been described previously; see for example, U.S. Pat. No. 6,048,530 dated Apr. 11, 2000.

2.3. α2-Macroglobulin

The α-macroglobulins are members of a protein superfamily of structurally related proteins which also comprises complement components C3, C4 and C5. The human plasma protein alpha(2)macroglobulin (α2M) is a 720 kDa homotetrameric protein primarily known as proteinase inhibitor and plasma and inflammatory fluid proteinase scavenger molecule (for review see Chu and Pizzo, 1994, Lab. Invest. 71:792). Alpha (2) macroglobulin is synthesized as a 1474 amino acid precursor, the first 23 of which function as a signal sequence that is cleaved to yield a 1451 amino acid mature protein (Kan et al., 1985, Proc. Natl. Acad. Sci. U.S.A. 82:2282-2286).

Alpha(2)macroglobulin promiscuously binds to proteins and peptides with nucleophilic amino acid side chains in a covalent manner (Chu et al., 1994, Ann. N.Y. Acad. Sci. 737:291-307) and targets them to cells which express the α2M receptor (α2MR) (Chu and Pizzo, 1993, J. Immunol. 150:48). Binding of α2M to the α2MR is mediated by the C-terminal portion of α2M (Holtet et al., 1994, FEBS Lett. 344:242-246) and key residues have been identified (Nielsen et al., 1996, J. Biol. Chem. 271:12909-12912).

Generally known for inhibiting protease activity, α2M binds to a variety of proteases thorough multiple binding sites (see, e.g., Hall et al., 1981, Biochem. Biophys. Res. Commun.100(1):8-16). Protease interaction with α2M results in a complex structural rearrangement called transformation, which is the result of a cleavage within the “bait” region of α2M after the proteinase becomes “trapped” by thioesters. The conformational change exposes residues required for receptor binding, allowing the α2M-proteinase complex to bind to the α2MR. Methylamine can induce similar conformational changes and cleavage as that induced by proteinases. The uncleaved form of α2M, which is not recognized by the receptor, is often referred to as the “slow” form (s-α2M). The cleaved form is referred to as the “fast” form (f-α2M) (reviewed by Chu et al., 1994, Ann. N.Y. Acad. Sci. 737:291-307).

Studies have shown that, in addition to its proteinase-inhibitory functions, α2M, when complexed to antigens, can enhance the antigens' ability to be taken up by antigen presenting cells such as macrophages and presented to T cell hybridomas in vitro by up to two orders of magnitude (Chu and Pizzo, 1994, Lab. Invest. 71:792), and induce T cell proliferation (Osada et al., 1987, Biochem. Biophys. Res. Commun.146:26-31). Further evidence suggests that complexing antigen with α2M enhances antibody production by crude spleen cells in vitro (Osada et al., 1988, Biochem. Biophys. Res. Commun. 150:883) and elicits an in vivo antibody responses in experimental rabbits (Chu et al., 1994, J. Immunol. 152:1538-1545) and mice (Mitsuda et al., 1993, Biochem. Biophys. Res. Commun. 101:1326-1331). However, none of these studies have shown whether α2M-antigen complexes are capable of eliciting cytotoxic T cell responses in vivo.

α2M can form complexes with antigens, which are taken up by antigen presenting cells (“APCs”) via the α2MR, also known as LDL (low-density lipoprotein) Receptor-Related Protein (“LRP”) or CD91 (see provisional patent application No. 60/209,266 filed Jun. 2, 2000, which is incorporated by reference herein in its entirety). α2M directly competes for the binding of heat shock protein gp96 to the α2MR, indicating that α2M and hsps may bind to a common recognition site on the α2MR (Binder et al., 2000, Nature Immunology 1(2), 151-154). Additionally, α2M-antigenic peptide complexes prepared in vitro can be administered to animals to generate a cytotoxic T cell response specific to the antigenic molecules (Binder et al., 2001, J. Immunol. 166:4968-72). Thus, because hsps and α2M have a number of common functional attributes, such as the ability to bind peptide, the recognition and uptake by the α2MR, and the stimulation of a cytotoxic T cell response, α2M can be used for immunotherapy against cancer and infectious disease.

2.4. Immune Responses

An organism's immune system reacts with two types of responses to pathogens or other harmful agents—humoral response and cell-mediated response (See Alberts, B. et al., 1994, Molecular Biology of the Cell. 1195-96). When resting B cells are activated by antigen to proliferate and mature into antibody-secreting cells, they produce and secrete antibodies with a unique antigen-binding site. This antibody-secreting reaction is known as the humoral response. On the other hand, the diverse responses of T cells are collectively called cell-mediated immune reactions. There are two main classes of T cells-cytotoxic T cells and helper T cells. Cytotoxic T cells directly kill cells that are infected with a virus or some other intracellular microorganism. Helper T cells, by contrast, help stimulate the responses of other cells: they help activate macrophages, dendritic cells and B cells, for example (See Alberts, B. et al., 1994, Molecular Biology of the Cell. 1228). Both cytotoxic T cells and helper T cells recognize antigen in the form of peptide fragments that are generated by the degradation of foreign protein antigens inside the target cell, and both, therefore, depend on major histocompatibility complex (MHC) molecules, which-bind these peptide fragments, carry them to the cell surface, and present them there to the T cells (See Alberts, B. et al., 1994, Molecular Biology of the Cell. 1228). MHC molecules are typically found in abundance on antigen-presenting cells (APCs).

2.5. Antigen Presentation

Antigen-presenting cells (APCs), such as macrophages and dendritic cells, are key components of innate and adaptive immune responses. Antigens are generally ‘presented’ to T cells or B cells on the surfaces of other cells, the APCs. APCs can trap lymph- and blood-borne antigens and, after internalization and degradation, present antigenic peptide fragments, bound to cell-surface molecules of the major histocompatibility complex (MHC), to T cells. APCs may then activate T cells (cell-mediated response) to clonal expansion, and these daughter cells may either develop into cytotoxic T cells or helper T cells, which in turn activate B (humoral response) cells with the same MHC-bound antigen to clonal expansion and specific antibody production (See Alberts, B. et al., 1994, Molecular Biology of the Cell. 1238-45).

Two types of antigen-processing mechanisms have been recognized. The first type involves uptake of proteins through endocytosis by APCs, antigen fragmentation within vesicles, association with class II MHC molecules and expression on the cell surface. This complex is recognized by helper T cells expressing CD4. The other is employed for proteins, such as viral antigens, that are synthesized within the cell and appears to involve protein fragmentation in the cytoplasm. Peptides produced in this manner become associated with class I MHC molecules and are recognized by cytotoxic T cells expressing CD8 (See Alberts, B. et al., 1994, Molecular Biology of the Cell. 1233-34).

Stimulation of T cells involves a number of accessory molecules expressed by both T cell and APC. Co-stimulatory molecules are those accessory molecules that promote the growth and activation of the T cell, e.g., B7-1, B7-2, CD40, ICAM-1 and MHC II on the APC surface and CD28, CD40L, T-cell antigen surface receptors (TCRs) and CD4 on the T cell surface (See e.g., Banchereau and Steinman, 1998, Nature 392:245-252). Upon stimulation, co-stimulatory molecules induce release of cytokines, such as interleukin 1 (IL-1) or interleukin 2 (IL-2), interferon, etc., which promote T cell growth and expression of surface receptors (See e.g., Paul, 1989, Fundamental Immunology. 109-10).

### 3. SUMMARY OF THE INVENTION

The present invention provides a method of making a composition, which composition is immunogenic against a type of cancer or an agent of infectious disease, comprising mixing (i) an amount of a purified first complex (“Specific Complex”) comprising a first heat shock protein (“Specific hsp”) complexed to a first peptide which displays antigenicity of an antigen of said type of cancer or antigenicity of an antigen of an agent of said infectious disease; or an amount of a purified population of heterogeneous first complexes, said population of heterogeneous first complexes comprising a plurality of different first peptides, and (ii) an equal or greater amount of a second heat shock protein (“Non-Specific hsp”), which second heat shock protein is not complexed in vitro to a peptide which displays antigenicity of an antigen of said type of cancer or antigenicity of an antigen of an agent of said infectious disease, and which is not in the form of a complex, said complex having been isolated as a complex from cancerous tissue of said type of cancer or cells infected with said agent of infectious disease, respectively. In one embodiment, each first heat shock protein in the population of heterogeneous first complexes is bound to a different first peptide

Alternatively, the present invention provides methods of making a composition, which composition is immunogenic against a type of cancer or an agent of infectious disease, comprising mixing (i) an amount of a purified first complex (“Specific Complex”) comprising a first heat shock protein (“Specific hsp”) complexed to a first peptide which displays antigenicity of an antigen of said type of cancer or antigenicity of an antigen of an agent of said infectious disease; or an amount of a purified population of heterogeneous first complexes, said population of heterogeneous first complexes comprising a plurality of different first peptides, and (ii) an equal or greater amount of α2M (“Non-Specific α2M). In one embodiment, each first heat shock protein in said population of heterogeneous first complexes is bound to a different first peptide. In a preferred embodiment, the α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

Alternatively, the present invention provides methods of making a composition, which composition is immunogenic against a type of cancer or an agent of infectious disease, comprising mixing (i) an amount of a purified first complex (“Specific Complex”) comprising a first α2M (“Specific α2M”) complexed to a first peptide which displays antigenicity of an antigen of said type of cancer or antigenicity of an antigen of an agent of said infectious disease; or an amount of a purified population of heterogeneous first complexes, said population of heterogeneous first complexes comprising a plurality of different first peptides, and (ii) an equal or greater amount of a second α2M (“Non-Specific α2M). In a preferred embodiment, the second α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In yet alternative embodiments, the present invention provides a method of making a composition, which composition is immunogenic against a type of cancer or an agent of infectious disease, comprising mixing (i) an amount of a purified first complex (“Specific Complex”) comprising α2M (“Specific α2M”) complexed to a first peptide which displays antigenicity of an antigen of said type of cancer or antigenicity of an antigen of an agent of said infectious disease, and (ii) an equal or greater amount of a heat shock protein (“Non-Specific hsp”). In a preferred embodiment, the heat shock protein is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

The present invention further provides a method of making a composition, which composition is immunogenic against a type of cancer or an agent of infectious disease, said method comprising purifying a first heat shock protein-peptide complex (“Specific Complex,” the hsp component of which is the “Specific hsp”) from cancerous tissue of said type of cancer or metastasis thereof, or cells infected with said agent of infectious disease, and mixing an amount of said first complex with an equal or greater amount of a second heat shock protein (“Non-Specific hsp”), which second heat shock protein is not complexed in vitro to a peptide which displays antigenicity of an antigen of said type of cancer or antigenicity of an antigen of an agent of said infectious disease, and which is not in the form of a complex, said complex having been isolated as a complex from cancerous tissue of said type of cancer or cells infected with said agent of infectious disease, respectively.

Alternatively, the present invention provides a method of making a composition, which composition is immunogenic against a type of cancer or an agent of infectious disease, said method comprising purifying a first heat shock protein-peptide complex (“Specific Complex,” the hsp component of which is the “Specific hsp”) from cancerous tissue of said type of cancer or metastasis thereof, or cells infected with said agent of infectious disease, and mixing an amount of said first complex with an equal or greater amount of α2M (“Non-Specific α2M”). In a preferred embodiment, the α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

Alternatively, the present invention provides a method of making a composition, which composition is immunogenic against a type of cancer or an agent of infectious disease, said method comprising purifying a first α2M-peptide complex (“Specific Complex,” the α2M component of which is the “Specific α2M”) from cancerous tissue of said type of cancer or metastasis thereof, or cells infected with said agent of infectious disease, and mixing an amount of said first complex with an equal or greater amount of a second α2M (“Non-Specific α2M”). In a preferred embodiment, the second α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In yet alternative embodiments, the present invention further provides a method of making a composition, which composition is immunogenic against a type of cancer or an agent of infectious disease, said method comprising purifying an α2M-peptide complex (“Specific Complex,” the α2M component of which is the “Specific α2M”) from cancerous tissue of said type of cancer or metastasis thereof, or cells infected with said agent of infectious disease, and mixing an amount of said first complex with an equal or greater amount of a heat shock protein (“Non-Specific hsp”). In a preferred embodiment, the heat shock protein is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

The present invention further provides a method of making a composition, which composition is immunogenic against a type of cancer or an agent of infectious disease, said method comprising complexing in vitro a first heat shock protein (“Specific hsp”) to a tumor-specific antigen of said type of cancer or an antigen of said agent of said infectious disease, to produce a first complex (“Specific Complex”), and mixing an amount of said first complex with an equal or greater amount of a second heat shock protein (“Non-Specific hsp”) that is not complexed in vitro to a peptide which displays antigenicity of an antigen of said type of cancer or antigenicity of an antigen of an agent of said infectious disease, and which second heat shock protein is not in the form of a complex, said complex having been isolated as a complex from cancerous tissue of said type of cancer or cells infected with said agent of infectious disease, respectively.

Alternatively, the present invention further provides a method of making a composition, which composition is immunogenic against a type of cancer or an agent of infectious disease, said method comprising complexing in vitro a first heat shock protein (“Non-Specific hsp”) to a tumor-specific antigen of said type of cancer or an antigen of said agent of said infectious disease, to produce a first complex (“Specific Complex”), and mixing an amount of said first complex with an equal or greater amount of a α2M (“Non-Specific α2M”). In a preferred embodiment, the α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

Alternatively, the present invention further provides a method of making a composition, which composition is immunogenic against a type of cancer or an agent of infectious disease, said method comprising complexing in vitro a first α2M (“Non-Specific αC2M”) to a tumor-specific antigen of said type of cancer or an antigen of said agent of said infectious disease, to produce a first complex (“Specific Complex”), and mixing an amount of said first complex with an equal or greater amount of a second α2M (“Non-Specific α2M”). In a preferred embodiment, the second α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

The present invention further provides a method of making a composition, which composition is immunogenic against a type of cancer or an agent of infectious disease, said method comprising complexing in vitro an α2M (“Specific α2M”) to a tumor-specific antigen of said type of cancer or an antigen of said agent of said infectious disease, to produce a first complex (“Specific Complex”), and mixing an amount of said first complex with an equal or greater amount of a heat shock protein (“Non-Specific hsp”). In a preferred embodiment, the heat shock protein is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

The present invention further provides a composition made by mixing (i) an amount of a purified first complex comprising a first heat shock protein (“Specific hsp”) complexed to a peptide which displays antigenicity of an antigen of said type of cancer or antigenicity of an antigen of an agent of said infectious disease (said complex being the “Specific Complex”), and (ii) an equal or greater amount of a second heat shock protein (“Non-Specific hsp”) that is not complexed in vitro to a peptide which displays antigenicity of an antigen of said type of cancer or antigenicity of an antigen of an agent of said infectious disease, and is not in the form of a complex, said complex having been isolated as a complex from cancerous tissue of said type of cancer or cells infected with said agent of infectious disease, respectively.

In alternative embodiments, the present invention further provides a composition made by mixing (i) an amount of a purified first complex comprising a first heat shock protein (“Specific hsp”) complexed to a peptide which displays antigenicity of an antigen of said type of cancer or antigenicity of an antigen of an agent of said infectious disease (said complex being the “Specific Complex”), and (ii) an equal or greater amount of (α2M (“Non-Specific α2M”). In a preferred embodiment, the α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In alternative embodiments, the present invention further provides a composition made by mixing (i) an amount of a purified first complex comprising a first α2M (“Specific α2M”) complexed to a peptide which displays antigenicity of an antigen of said type of cancer or antigenicity of an antigen of an agent of said infectious disease (said complex being the “Specific Complex”), and (ii) an equal or greater amount of a second α2M (“Non-Specific α2M”). In a preferred embodiment, the second α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In yet other alternative embodiments, the present invention further provides a composition made by mixing (i) an amount of a purified first complex comprising an α2M (“Specific α2M”) complexed to a peptide which displays antigenicity of an antigen of said type of cancer or antigenicity of an antigen of an agent of said infectious disease (said complex being the “Specific Complex”), and (ii) an equal or greater amount of a heat shock protein (“Non-Specific hsp”). In a preferred embodiment, the heat shock protein is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

The present invention yet further provides a composition comprising a purified first complex comprising (i) a first heat shock protein (“Specific hsp”) complexed to a peptide which displays antigenicity of an antigen of a type of cancer or antigenicity of an antigen of an agent of an infectious disease (said complex being the Specific Complex”), and (ii) a second heat shock protein (“Non-Specific hsp”) that is not complexed in vitro to a peptide which displays antigenicity of an antigen of said type of cancer or antigenicity of an antigen of an agent of said infectious disease, and is not in the form of a complex, said complex having been isolated as a complex from cancerous tissue of said type of cancer or cells infected with said agent of infectious disease, wherein the amount of the first heat shock protein is less than or equal to the second heat shock protein, and wherein the composition is immunogenic against said type of cancer or said agent of infectious disease, respectively.

In alternative embodiments, the present invention yet further provides a composition comprising a purified first complex comprising (i) a first heat shock protein (“Specific hsp”) complexed to a peptide which displays antigenicity of an antigen of a type of cancer or antigenicity of an antigen of an agent of an infectious disease (said complex being the “Specific Complex”) and (ii) an (α2M (“Non-Specific α2M”), wherein the amount of the first heat shock protein is less than or equal to the α2M, and wherein the composition is immunogenic against said type of cancer or said agent of infectious disease, respectively. In a preferred embodiment, the α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In alternative embodiments, the present invention yet further provides a composition comprising a purified first complex comprising (i) a first α2M (“Specific α2M”) complexed to a peptide which displays antigenicity of an antigen of a type of cancer or antigenicity of an antigen of an agent of an infectious disease (said complex being the “Specific Complex”) and (ii) a second α2M (“Non-Specific α2M”), wherein the amount of the first α2M is less than or equal to the second α2M, and wherein the composition is immunogenic against said type of cancer or said agent of infectious disease, respectively. In a preferred embodiment, the α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In yet alternative embodiments, the present invention yet further provides a composition comprising a purified first complex comprising (i) an α2M (“Specific α2M”) complexed to a peptide which displays antigenicity of an antigen of a type of cancer or antigenicity of an antigen of an agent of an infectious disease (said complex being the Specific Complex”), and (ii) a heat shock protein (“Non-Specific hsp”). In a preferred embodiment, the heat shock protein is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

The present invention further provides methods of eliciting an immune response against a type of cancer or against an agent of an infectious disease in an individual, comprising administering to the individual an amount of a purified composition effective to elicit an immune response against said type of cancer or said agent of infectious disease, said composition comprising (i) an amount of a purified first complex (“Specific Complex”) comprising a first heat shock protein (“Specific hsp”) complexed to a peptide which displays antigenicity of an antigen of said type of cancer or antigenicity of an antigen of an agent of said infectious disease, and (ii) an equal or greater amount of α2M (“Non-Specific α2M”). In a preferred embodiment, the α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

The present invention further provides methods of eliciting an immune response against a type of cancer or against an agent of an infectious disease in an individual, comprising administering to the individual an amount of a purified composition effective to elicit an immune response against said type of cancer or said agent of infectious disease, said composition comprising (i) an amount of a purified first complex (“Specific Complex”) comprising a first α2M (“Specific α2M”) complexed to a peptide which displays antigenicity of an antigen of said type of cancer or antigenicity of an antigen of an agent of said infectious disease, and (ii) an equal or greater amount of a second α2M (“Non-Specific α2M”). In a preferred embodiment, the second α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

The present invention further provides methods of eliciting an immune response against a type of cancer or against an agent of an infectious disease in an individual, comprising administering to the individual an amount of a purified composition effective to elicit an immune response against said type of cancer or said agent of infectious disease, said composition comprising (i) an amount of a purified first complex (“Specific Complex”) comprising a first heat shock protein (“Specific hsp”) complexed to a peptide which displays antigenicity of an antigen of said type of cancer or antigenicity of an antigen of an agent of said infectious disease, and (i) an equal or greater amount of a second heat shock protein (“Non-Specific hsp”) that is not complexed in vitro to a peptide which displays antigenicity of an antigen of said type of cancer or antigenicity of an antigen of an agent of said infectious disease, respectively, and is not in the form of a complex, said complex having been isolated as a complex from cancerous tissue of said type of cancer or cells infected with said agent of infectious disease, respectively.

In alterative embodiments, the present invention further provides methods of eliciting an immune response against a type of cancer or against an agent of an infectious disease in an individual, comprising administering to the individual an amount of a purified composition effective to elicit an immune response against said type of cancer or said agent of infectious disease, said composition comprising (i) an amount of a purified first complex (“Specific Complex”) comprising a first heat shock protein (“Specific hsp”) complexed to a peptide which displays antigenicity of an antigen of said type of cancer or antigenicity of an antigen of an agent of said infectious disease, and (ii) an equal or greater amount of α2M “Non-Specific α2M”). In a preferred embodiment, the α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In alterative embodiments, the present invention further provides methods of eliciting an immune response against a type of cancer or against an agent of an infectious disease in an individual, comprising administering to the individual an amount of a purified composition effective to elicit an immune response against said type of cancer or said agent of infectious disease, said composition comprising (i) an amount of a purified first complex (“Specific Complex”) comprising a first α2M (“Specific α2M”) complexed to a peptide which displays antigenicity of an antigen of said type of cancer or antigenicity of an antigen of an agent of said infectious disease, and (ii) an equal or greater amount of a second α2M (“Non-Specific α2M”). In a preferred embodiment, the second α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In yet alternative embodiments, the present invention further provides methods of eliciting an immune response against a type of cancer or against an agent of an infectious disease in an individual, comprising administering to the individual an amount of a purified composition effective to elicit an immune response against said type of cancer or said agent of infectious disease, said composition comprising (i) an amount of a purified first complex (“Specific Complex”) comprising an α2M (“Specific α2M”) complexed to a peptide which displays antigenicity of an antigen of said type of cancer or antigenicity of an antigen of an agent of said infectious disease, and (i) an equal or greater amount of a heat shock protein (“Non-Specific hsp”). In a preferred embodiment, the heat shock protein is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

The present invention further provides methods of treating or preventing a type of cancer or an infectious disease in an individual in whom said treatment or prevention is desired, comprising administering to the individual a therapeutically effective amount of a purified composition, said composition comprising (i) an amount of a purified first complex (“Specific Complex”) comprising a first heat shock protein (“Specific hsp”) complexed to a peptide which displays antigenicity of an antigen of said type of cancer or antigenicity of an antigen of an agent of said infectious disease, and (ii) an equal or greater amount of a second heat shock protein (“Non-Specific hsp”) that is not complexed in vitro to a peptide which displays antigenicity of an antigen of said type of cancer or antigenicity of an antigen of an agent of said infectious disease, respectively, and is not in the form of a complex, said complex having been isolated as a complex from cancerous tissue of said type of cancer or cells infected with said agent of infectious disease, respectively.

In yet alternative embodiments, the present invention further provides methods of treating or preventing a type of cancer or an infectious disease in an individual in whom said treatment or prevention is desired, comprising administering to the individual a therapeutically effective amount of a purified composition, said composition comprising (i) an amount of a purified first complex (“Specific Complex”) comprising an α2M (“Specific α2M”) complexed to a peptide which displays antigenicity of an antigen of said type of cancer or antigenicity of an antigen of an agent of said infectious disease, and (ii) an equal or greater amount of a heat shock protein (“Non-Specific hsp”). In a preferred embodiment, the heat shock protein is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In an alternative embodiment, the present invention further provides methods of treating or preventing a type of cancer or an infectious disease in an individual in whom said treatment or prevention is desired, comprising administering to the individual a therapeutically effective amount of a purified composition, said composition comprising (i) an amount of a purified first complex (“Specific Complex”) comprising a first heat shock protein (“Specific hsp”) complexed to a peptide which displays antigenicity of an antigen of said type of cancer or antigenicity of an antigen of an agent of said infectious disease, and (ii) an equal or greater amount of α2M (“Non-Specific α2M”). In a preferred embodiment, the α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In an alternative embodiment, the present invention further provides methods of treating or preventing a type of cancer or an infectious disease in an individual in whom said treatment or prevention is desired, comprising administering to the individual a therapeutically effective amount of a purified composition, said composition comprising (i) an amount of a purified first complex (“Specific Complex”) comprising a first α2M (“Specific α2M”) complexed to a peptide which displays antigenicity of an antigen of said type of cancer or antigenicity of an antigen of an agent of said infectious disease, and (ii) an equal or greater amount of a second α2M (“Non-Specific α2M”). In a preferred embodiment, the second α2M is not complexed in vitro to a peptide which displays antigenicity of an antigen of said type of cancer or antigenicity of an antigen of an agent of said infectious disease, and/or is not in the form of a complex, said complex having been isolated as a complex from cancerous tissue of said type of cancer or cells infected with said agent of infectious disease, respectively.

In certain specific embodiments of the foregoing methods and compositions, the mass ratio of the Specific hsp or Specific α2M to the Non-Specific hsp or to the Non-Specific α2M is 1:1, 1:2, more preferably 1:5, and most preferably 1:10. In other embodiments, the mass ratio of the Specific hsp or Specific α2M to Non-Specific hsp or to the Non-Specific α2M is 1:100, 1:500 or 1:1,000. In yet other embodiments, the ratio of the Specific hsp or Specific α2M to Non-Specific hsp or to the Non-Specific α2M is 1:3, 1:4, 1:9, 1:19, 1:24, 1:49 or 1:99.

In one embodiment of the foregoing methods and compositions, the Non-Specific hsp or the Non-Specific α2M is not complexed to any molecule. In other embodiments, the Non-Specific hsp or the Non-Specific (α2M is complexed to a second peptide to produce a second complex (the “Non-Specific Complex” or the “Diluent Complex”).

In a specific embodiment of the foregoing methods and compositions, the Specific hsp and the Non-Specific hsp can be the same.

In a preferred embodiment of the foregoing methods and compositions, where a Specific Complex comprising α2M is isolated from a liver cancer cell, the Non-Specific hsp is not isolated from the cell from which the Specific Complex is isolated (e.g., a cell with a genotype that is the same as the cell from which the Specific Complex is isolated).

In certain specific embodiments of the foregoing methods and compositions, the Non-Specific hsp or the Non-Specific α2M can be present in a cell lysate or extract that is mixed with the Specific Complex. A lysate comprising α2M is preferably prepared from liver cells, and yet more preferably from a recombinant cell in culture that expresses α2M.

The components of the Specific Complex and/or the Non-Specific Complex of the foregoing methods and compositions (i.e., the hsp or α2M and its complexed antigenic molecule) can be covalently or noncovalently linked. Preferably, the Specific Complex and/or the Non-Specific Complex is purified to apparent homogeneity, as detected on a SDS-PAGE gel.

In certain embodiments of the methods disclosed hereinabove, a dosage of the amount of the Specific Complex in the composition for eliciting an immune response or for prevention or treatment of cancer or infectious disease is in the range of 0.1 to 2 micrograms. In other embodiments, the amount of the Specific Complex composition is in the range of 5 to 20 micrograms. In certain specific embodiments, the amount of the Specific Complex in the composition is in the range of 0.1 to 2 micrograms and the mass ratio of the first heat shock protein or α2M to the second heat shock protein or α2M is 1:10. In a preferred mode of these embodiments, the first heat shock protein is hsp70 or gp96.

In yet other specific embodiments, the amount of the First Complex in the composition is in the range of 5 to 20 micrograms and the mass ratio of the Specific heat shock protein or α2M to the Non-Specific heat shock protein or α2M is 1:10. In one mode of these embodiments, the Specific heat shock protein is hsp90. In another mode of the embodiment, the Specific heat shock protein is hsp70or gp96.

In other embodiments of the methods disclosed hereinabove, a dosage of the amount of the Diluted Complex in the composition for eliciting an immune response or for prevention or treatment of cancer or infectious disease is 1-100 μg, more preferably 2-50 μg, and is most preferably about 5-25 μg where the Specific Complex comprises gp96, hsp70, hsp 110 or grp170. Where the Specific Complex comprises hsp90, the dosage of Diluted Complex is preferably 10-500 μg, more preferably 20-400 μg, and yet more preferably 50-250 μg. In other embodiments, where the Specific Complex comprises calreticulin, the dosage of Diluted Complex is preferably 0.5-50 μg, more preferably 1-25 μg, yet more preferably 2 μg-15 μg, and is most preferably 2.5-10 μg. Where the Specific complex comprises α2M, the dosage of Diluted Complex is preferably 1 μg-10 mg, more preferably 2 μg-5 mg, more preferably 5 μg-500 μg, and is most preferably 5-250 μg.

In one embodiment in which eliciting an immune response against or the treatment or prevention of a type of cancer is desired, the first complex is prepared from cancerous tissue of said type of cancer or a metastasis thereof autologous to the individual. In another embodiment in which eliciting an immune response against or the treatment or prevention of a type of cancer is desired, the first complex is prepared from cancerous tissue of said type of cancer or a metastasis thereof allogeneic to the individual.

### 4. DETAILED DESCRIPTION OF THE INVENTION

The present invention relates to the improvement of efficiency of vaccinations with heat shock protein preparations. In particular, the invention provides novel formulations of heat shock/stress protein-peptide complexes. Methods of use of the formulations for the prevention and treatment of cancer and infectious diseases, and for eliciting an immune response in a subject, are also provided. The invention is useful in situations when the supply of hsp-peptide complexes isolated from an antigen source, such as cancer tissues or infected tissues, is limited in supply. The amount of hsp-peptide complex from a tumor source is often too limiting to allow for a full course of immunotherapy, see e.g., Lewis et al., 1999, Proceedings of ASCO 18, abstract no. 1687.

While not bound by any theory, the invention is based, in part, on the recognition that, in the amount of hsp-peptide complexes-based vaccine currently used for the treatment or prevention of cancer or infectious disease, there is an abundance of the antigenic peptides that stimulate the recipient's immune system resulting in an immune response against the cancer or infectious disease. Vaccination of mice with 10 μg gp96-peptide preparation from a tumor renders the mice resistant to the tumor (Srivastava et al., 1986, Proc. Natl. Acad. Sci. USA 83:3407-3411). Assuming the molecular weights of the peptides are negligible, a preparation of 10 μg gp96-peptide complexes contains approximately 6×10¹³ molecules of gp96, as calculated from Avogadro's number. Assuming that equimolar quantities of hsps and peptides are present in a given preparation, approximately 6×10¹³ molecules of peptides will be present in this preparation. Of these, the inventors estimated that about 0.01% of the peptides are antigenic peptides that are specific to the antigen source. Accordingly, when 10 μg of a gp96-peptide preparation is used in vaccination, it contains approximately 10⁹ source-specific antigenic peptides.

In an immune response, after contact with an antigen presented by an antigen presenting cell (APC), T cell recptors (TCRs) are down-regulated from the T cell surface by internalization. It is generally thought that after a TCR is down-regulated, an antigen on the surface of an APC is then free to engage another TCR. A single antigen, in the context of antigen presentation by the MHC, may serially engage up to 200 TCRs (Valutti et al., 1995, Nature 375:148-151). Further, it has been demonstrated that maximal activation of a T cell occurs upon engagement of approximately 8000 TCRs in the absence of costimulatory molecules, or approximately 1500 TCRs in the presence of costimulatory molecules (Viola and Lanzavecchia, 1996, Science 273:104-106).

Assuming that only 1% of the 10⁹ calculated peptides are channeled productively by APCs to be presented at the cell surface, then 10⁷ peptides are effectively presented following the administration of 10 μg hsp-peptide complex. If these peptides are presented by 10⁵ APCs, then each APC will present approximately 100 antigenic peptides. Each antigenic molecule may engage a T cell receptor up to 200 times. If 100 antigenic peptides are presented per APC, each of which can engage a receptor up to 200 times, up to 20,000 receptor engagement events may take place per T cell following administration of 10 μg hsp-peptide complex. As T cell stimulation requires only the engagement of approximately 1500 T cell receptors in the presence of costimulatory molecules, the administration of 10 μg hsp-peptide complex is a potent stimulus for T cell stimulation particularly in the presence of costimulatory molecules. Specifically, according to the inventor's calculations, only 1500, or 7.5%, of the 20,000 TCR engagement events that may take place per T cell following administration of 10 μg hsp-peptide complex are required to elicit an immune response in a subject. Accordingly, only approximately 10% of a composition comprising 10 μg hsp-peptide complex (or an α2M-peptide complex of a comparable molecular mass) need be isolated from a source containing antigenic peptides. The remainder of the dose can comprise Diluent, or Non-Specific, hsps, hsp-peptide complexes, α2M, α2M-peptide complexes. Diluents include, but are not limited to, cell extracts or lysates comprising non-specific hsp-peptide or α2M-peptide complexes, hsps or α2M. Thus, for example, approximately 1 μg of gp 96 purified from tumor cells can be mixed with approximately 9 μg of gp96 purified from normal tissue to yield a composition in the total amount of 10 μg.

In one embodiment of the invention, the immunogenic compositions of the invention are formulated by mixing (i) an initial amount of a preparation of hsp-peptide complexes or α2M-peptide complexes that comprises antigenic peptides specific to an antigenic source of interest, and (ii) a preparation of hsp, α2M, hsp-peptide complexes or α2M-peptide complexes that does not comprise significant amounts of antigenic peptides specific to the antigen source of interest, such that the number of immunogenic administrations that can be made with the initial amount of the preparation is increased. In effect, by the methods of formulation of the invention, the preparation of hsp-peptide or α2M-peptide complexes that comprises antigenic peptides is “Diluted” without reducing the ability of the resulting hsp-peptide or α2M-peptide complexes to elicit, stimulate, enhance or sustain a specific immune response in vivo or in vitro. Further, a Diluted Complex may possess greater immunogenicity or antigenicity than an undiluted preparation comprising an equal amount of the corresponding Specific Complex.

Accordingly, the methods of the invention comprise methods of eliciting an immune response in an individual in whom the treatment or prevention of cancer or infectious disease is desired, by administering, by any route, preferably subcutaneously, more preferably intradermally, a composition comprising an amount of a first, “Specific” complex consisting essentially of hsps or α2M bound to antigenic molecules effective to elicit an immune response against tumor cells or an agent of an infectious disease, and a “Diluent.” The Diluent can be an hsp, hsp complexed to a molecule that is not a specific antigenic peptide, α2M, or α2M complexed to an antigenic molecule that is preferably not a specific antigenic peptide. Accordingly, the Diluent may comprise an uncomplexed hsp, α2M, or, in another embodiment, an hsp or α2M complexed to another molecule, including but not limited to a peptide. The Diluent can also be a cell extract or lysate comprising hsps or α2M. The Diluent is present in the composition, referred to as a “Diluted Complex”, in amount that is equal in mass or moles to the Specific Complex, and is more preferably in excess of the Specific Complex (in either mass or moles). The amount of the Diluted Complex administered will vary depending on the amount of Specific Complex in the Diluted Complex. A dosage can be measured in terms of the Diluted Complex or in terms of the Specific Complex component of the Diluted complex. The dosage of Diluted Complex is preferably 1-100 μg where the Specific Complex comprises gp96 or hsp70, and is more preferably 2-50 μg, and yet most preferably about 5-25 μg. Where the Specific Complex comprises hsp90, the dosage of Diluted Complex is preferably 10-500 μg, more preferably 20-400 μg, and yet more preferably 50-250 μg. In other embodiments, a dosage of Diluted Complex comprises 1, 2, 5 or 10 μg of a Specific Complex comprising gp96 or hsp 70, regardless of the total amount of Diluted Complex. In yet other embodiments, a dosage of Diluted Complex comprises 10, 20, 50 or 100 μg of a Specific Complex comprising hsp 90, regardless of the total amount of Diluted Complex. Additional dosages are described in § 4.13.1, supra.

The hsps that can be used for the practice of the present invention, in both the Specific Complexes and in the Diluents include but are not limited to, hsp70, hsp90, gp96, calreticulin, hsp 110, grp170, alone or in combination. Preferably, the hsps are human hsps. The hsps of the Specific Complexes and Diluents can be the same or different hsps.

The α2M polypeptide or α2M-antigenic molecules complexes used in the practice of the present invention, in both the Specific Complexes and in the Diluents, can be expressed recombinantly (for example, as described in §§ 4.2.6 and 4.6.2). Alternatively, α2M polypeptide can be purchased commercially, or purified from tissue (e.g., liver tissue, where α2M is predominantly expressed) or blood.

In the practice of the invention, therapy by administration of hsp-peptide or α2M-peptide complexes using any convenient route of administration may optionally be in combination with adoptive immunotherapy involving the administration of antigen-presenting cells that have been sensitized in vitro with a Specific Complex that is optionally diluted with a Diluent Complex.

In a specific embodiment, the present invention relates to methods and compositions for prevention and treatment of primary and metastatic neoplastic diseases.

Specific therapeutic regimens, pharmaceutical compositions, and kits are provided by the invention.

As used herein, unless otherwise indicated, the terms “hsp”, “α2M” “complex”, when used in the singular, also encompasses a plurality of hsps, α2M proteins and a plurality of complexes of hsps and peptides or α2M and peptides, and may refer to a population of hsps, α2M, hsp-peptide complexes or α2M-peptide complexes.

As used herein, the term “Specific Complex” refers to a hsp-peptide or α2M-peptide complex that comprises an antigenic peptide specific to an antigen source of interest. “Specific Complexes” refers to a population of hsp-peptide or α2M-peptide complexes that comprise molecular complexes of hsps or α2M covalently or noncovalently associated with antigenic peptides specific to an antigen source of interest. The source of antigens depends on the purpose of the therapeutic and/or prophylactic application. Tumor tissues, tumor cells, cancer cells, or cells infected with a pathogen can be, without limitation, sources of antigenic peptides. An immunogenic amount of a Specific Complexes of the invention is capable of, through at least one administration, eliciting, stimulating, enhancing, and/or sustaining an immune response in a subject against antigenic peptides specific to an antigen source of interest.

As used herein, the term “Diluents” refers to hsps, α2M, and hsp- or α2M-molecular complexes. Where the Diluent comprises an hsp or α2M preparation, the hsp or α2M preparation preferably does not comprise any significant amounts of antigenic peptides specific to an antigen source of interest. Diluents may comprise hsps or α2M alone, or hsps or α2M covalently or noncovalently associated with other molecules, including peptides. In one embodiment, Diluents simply consist of purified, recombinantly expressed hsps or α2M. In another embodiment, the Diluent is an hsp-peptide or α2M-peptide complex prepared from a cell line. In yet another embodiment, the Diluents are hsp-peptide or α2M-peptide complexes prepared from normal (i.e., non-cancerous or uninfected) cells of the subject to whom the Diluted Complex is to be administered, and therefore comprise non-specific antigenic peptides that are present as non-antigenic peptide components of specific hsp-peptide or α2M-peptide complex populations prepared from cells that express the antigenic peptides of interest. In yet another embodiment, the Diluent is a cell extract or lysate from a cell which does not express significant levels of the antigenic peptides of interest. Diluents that comprise hsp-peptide or α2M-peptide complexes may also comprise a negligible amount of antigenic peptides specific to the antigen source of interest; it cannot, however, when administered by itself to a subject, elicit, stimulate, enhance, and/or sustain with specificity an immune response in the subject against antigenic peptides specific to an antigen source of interest.

Where the Specific Complex or Diluent/Non-Specific Complex is purified from a cell or cell line, the cell or cell line can recombinantly express the corresponding hsp or α2M, for example by transfection of the cell with an hsp or α2M expression construct under the control of the appropriate transcription and translation signals.

As used herein, the term “Diluted Complexes” refers to immunogenic hsp or α2M molecular complexes that result from mixing Diluents and Specific Complexes, according to the methods of formulation of the invention.

In certain specific embodiments, the invention provides Diluted Complexes comprising an immunogenic mixture of Specific Complexes and Diluents. The Diluted Complexes of the invention may comprise any mass ratio of the first hsp/α2M (i.e., Specific hsp/α2M) to the second hsp/α2M (i.e., the Non-Specific hsp/α2M), or of Specific Complexes to Diluents, e.g., 1:1, 1:2, 1:3, 1:4, 1:5, 1:9, 1:10, 1:24, 1:49, 1:50, 1:99, 1:100, 1:500, 1:1,000, etc.

According to the invention, an immunogenic administration of Specific Complexes or Diluted Complexes to a subject results in eliciting, stimulating, enhancing, and/or sustaining an immune response in the subject against antigenic peptides specific to an antigen source of interest. Each administration to the subject uses a dose of Specific Complexes or Diluted Complexes, that is immunogenic. Depending on the initial physical amounts of Specific Complexes, the resulting Diluted Complexes can be divided into multiple doses, each of which is immunogenic when administered. For example, an immunogenic amount of Specific Complexes that is sufficient only for one immunogenic administration can now be used in multiple administrations after it has been diluted according to the invention.

In yet another embodiment, the invention provides, for a therapeutic and/or prophylactic application, a pharmaceutical formulation or composition comprising a dose of Diluted Complexes that is useful for a single immunogenic administration. The immunogenic dose may differ for different subjects and different therapeutic or prophylactic applications.

In practice, the formulations of the invention comprise reduced amounts of Specific Complexes isolated from tumor tissues or pathogen-infected tissues per administration. Because a smaller amount of the Specific Complexes is used per administration, a larger number of immunogenic administrations can be made. The immunogenic administrations can be made over an extended period of time and/or at multiple sites on the same subject. The additional number of immunogenic administrations that can be made with a finite amount of a Specific Complexes improve the economics and the flexibility of the treatment regimen.

Accordingly, in another embodiment, the invention further provides kits comprising a plurality of containers each comprising a pharmaceutical formulation or composition comprising a dose of Diluted Complexes sufficient for a single immunogenic administration. The invention also provides kits comprising a container comprising an immunogenic amount of Specific Complexes, and a container comprising Diluents. Optionally, instructions for formulating the Specific Complexes according to the methods of the invention can be included in the kits.

In further embodiments, the invention provides methods of eliciting an immune response in a subject in whom the treatment or prevention of infectious diseases or cancer is desired by administering an immunogenic amount of Diluted Complexes, or a pharmaceutical formulation or composition thereof Preferably, the administration is made intradermally or subcutaneously.

In yet another embodiment, the methods of use of the pharmaceutical formulations or compositions of the invention may optionally be applied in combination with adoptive immunotherapy. The antigen-presenting cell (APC) can be selected from among those APCs known in the art, including but not limited to macrophages, dendritic cells, B lymphocytes, and a combination thereof, and are preferably dendritic cells. The APCs can be sensitized by using an effective amount of the Specific Complexes or Diluted Complexes. The hsp-peptide-sensitized or α2M-peptide-sensitized APCs may be administered concurrently or before or after administration of the hsp-peptide complexes. The Specific Complex can be the same or different from the hsp-peptide or α2M-peptide complex used to sensitize the APCs. In a specific embodiment wherein the APCs and the compositions of the invention are administered concurrently, the APCs and composition of the invention can be present in the same composition (comprising APCs, Specific Complex, and Non-Specific Complex; or APCs and Diluted Complex) or different composition. Adoptive immunotherapy according to the invention allows activation of immune antigen presenting cells by incubation with hsp-peptide or α2M-peptide complexes. Preferably, prior to use of the cells in vivo measurement of reactivity against the tumor or infectious agent in vitro is done. This in vitro boost followed by clonal selection and/or expansion, and patient administration constitutes a useful therapeutic/prophylactic strategy.

In a preferred embodiment, Specific Complexes of a composition of the invention in which the Specific Antigen displays the antigenicity of a cancer antigen are autologous to the individual to whom they are administered; that is, a Specific Complex is isolated from cells of the individual, which cells are either infected with an agent of infectious disease, or are precancerous, cancerous, including metastatic (e.g., the Specific Complexes are prepared from infected tissues or tumor biopsies of the patient). The Diluents can also be autologous to the individual, for example prepared or isolated from normal cells of the individual. In another embodiment, the Specific Complexes are produced in vitro (e.g., wherein a complex with an exogenous antigenic molecule is desired). Similarly, a Diluent comprising or consisting of an hsp-peptide complex or α2M-peptide complex can be generated in vitro, for example by recombinant production methods using a cloned hsp or α2M originally derived from the individual or from others. In a specific embodiment relating to the prevention or treatment of cancer, the hsps and/or α2M in both the Specific Complexes and in the Diluents are autologous to (derived from) the patient to whom they are administered. The hsps, α2M and/or antigenic molecules can be purified from natural sources, chemically synthesized, or recombinantly produced.

Exogenous antigens and fragments and derivatives thereof for use in complexing with hsps or α2M to generate the Specific Complexes can be selected from among those known in the art, as well as those readily identified by standard immunoassays known in the art, for example by their ability to bind antibody or MHC molecules (antigenicity) or to generate immune response (immunogenicity). Specific Complexes of hsps or α2M and antigenic molecules can be isolated from cancerous (including tumor cells or metastatic tissue) or precancerous tissue of a patient, or from a cancer cell line, or can be produced in vitro (as is necessary in the embodiment in which an exogenous antigen is used as the antigenic molecule). Where the complexes comprising α2M are purified from a cell or cell line, the cell or cell line preferably recombinantly expresses α2M.

In various embodiments, the invention provides combinations of compositions which enhance the immunocompetence of the host individual and elicit specific immunity against infectious agents or specific immunity against preneoplastic and neoplastic cells. The therapeutic regimens and pharmaceutical compositions of the invention are described below. These compositions have the capacity to prevent the onset and progression of infectious diseases and prevent the development of tumor cells and to inhibit the growth and progression of tumor cells indicating that such compositions can induce specific immunity against agents of infectious diseases and tumor cells.

Accordingly, the invention provides methods of preventing and treating cancer in an individual comprising administering compositions comprising Diluted Complexes, said Diluted Complexes comprising Specific Complexes of hsps or α2M and peptides and Diluents comprising hsps, α2M, or hsp- or α2M-peptide complexes, optionally in combination with APC sensitized by Specific Complexes. Administration of the Diluted Complexes, alone or with the sensitized APCs, stimulates the immunocompetence of the host individual and elicits specific immunity against the preneoplastic and/or neoplastic cells. As used herein, “preneoplastic” cell refers to a cell which is in transition from a normal to a neoplastic form; and morphological evidence, increasingly supported by molecular biologic studies, indicates that preneoplasia progresses through multiple steps. Non-neoplastic cell growth commonly consists of hyperplasia, metaplasia, or most particularly, dysplasia (for review of such abnormal growth conditions (See Robbins and Angell, 1976, Basic Pathology, 2d Ed., W. B. Saunders Co., Philadelphia, pp. 68-79). Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. Although preneoplastic lesions may progress to neoplasia, they may also remain stable for long periods and may even regress, particularly if the inciting agent is removed or if the lesion succumbs to an immunological attack by its host. Cancers which can be treated with the compositions of the present invention include, but are not limited to, human sarcomas and carcinomas. Human sarcomas and carcinomas are also responsive to adoptive immunotherapy by the hsp complex-sensitized APCs.

The therapeutic regimens of the invention and pharmaceutical compositions comprising Diluted Complexes may be used with additional immune response enhancers or biological response modifiers including, but not limited to, the cytokines IFN-α, IFN-γ, IL-2, IL-4, IL-6, TNF, or other cytokine affecting immune cells. In accordance with this aspect of the invention, the compositions of the invention are administered in combination therapy with one or more of these cytokines. In another embodiment, the compositions of the invention are administered with radiotherapy or one or more chemotherapeutic agents for the treatment of cancer.

In addition to cancer therapy, the compositions of the invention can be utilized for the prevention of a variety of cancers, e.g., in individuals who are predisposed as a result of familial history or in individuals with an enhanced risk to cancer due to environmental factors.

4.1. Therapeutic Compositions Comprising Purified Hsp-peptide Complexes or α2M-peptide Complexes, for Eliciting Immune Responses to Cancer or Infectious Disease, and for In Vitro Sensitization of APC

The compositions comprising Diluted Complexes are administered to elicit an effective specific immune response to the complexed antigenic molecules in the Specific Complexes (and not to the hsp, α2M or the molecules in the Diluents). In accordance with the methods described herein, each Specific Complex employed in a composition of the invention is preferably purified in the range of 60 to 100 percent of the total mg protein, or at least 70%, 80% or 90% of the total mg protein. In another embodiment, each Specific Complex is purified to apparent homogeneity, as assayed by sodium dodecyl sulfate-polyacrylamide gel electrophoresis.

In a preferred embodiment, non-covalent complexes of hsp70, hsp90, gp96, calreticulin, hsp 110, or grp 170 with peptides are prepared and purified postoperatively from tumor cells obtained from the cancer patient for use as Specific Complexes in the compositions of the invention.

In accordance with the methods described herein, immunogenic or antigenic peptides that are endogenously complexed to hsps or MHC antigens can be used as specific antigenic molecules. For example, such peptides may be prepared that stimulate cytotoxic T cell responses against different tumor antigens (e.g., tyrosinase, gp100, melan-A, gp75, mucins, etc.) and viral proteins including, but not limited to, proteins of immunodeficiency virus type I (HIV-I), human immunodeficiency virus type II (HIV-II), hepatitis type A, hepatitis type B, hepatitis type C, influenza, Varicella, adenovirus, herpes simplex type I (HSV-A), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus and polio virus. The antigenic peptides can be naturally complexed to hsps or α2M in vivo and the complexes isolated from cells, or alternatively, produced in vitro from purified preparations of each of hsps/α2M and antigenic molecules.

In another specific embodiment, antigens of cancers (e.g., tumors) or infectious agents (e.g., viral antigen, bacterial antigens, etc.) can be obtained by purification from natural sources, by chemical synthesis, or recombinantly, and, through in vitro procedures such as that described below, complexed to hsps or α2M.

In an embodiment wherein the Specific Complex to be used is a complex that is produced in vivo in cells, exemplary purification procedures such as described in Sections 4.2.1-4.2.5 below can be employed. Alternatively, in an embodiment wherein one wishes to use antigenic molecules by complexing to hsps in vitro, hsps can be purified for such use from the endogenous hsp-peptide complexes in the presence of ATP or low pH (or chemically synthesized or recombinantly produced). In an embodiment in which antigenic molecules are complexed to α2M in vitro, α2M can be recombinantly expressed and complexed covalently or non-covalently to the antigenic molecules according to the methods described in Section 4.2.6 below. The protocols described herein may be used to isolate Specific Complexes and Diluents from any eukaryotic cells for example, tissues, isolated cells, or immortalized eukaryote cell lines infected with a preselected intracellular pathogen, tumor cells or tumor cell lines.

4.2. Heat Shock Proteins

Heat shock proteins, which are also referred to interchangeably herein as stress proteins, useful in the practice of the instant invention can be selected from among any cellular protein that satisfies the following criteria. It is a protein whose intracellular concentration increases when a cell is exposed to a stressful stimuli, is capable of binding other proteins or peptides, it is capable of releasing the bound proteins or peptides in the presence of adenosine triphosphate (ATP) or low pH, and shows at least 35% homology with any cellular protein having any of the above properties.

The first stress proteins to be identified were the heat shock proteins (hsps). As their name implies, hsps are synthesized by a cell in response to heat shock. To date, five major classes of hsps have been identified, based on the molecular weight of the family members. These classes are called shsps (small heat shock proteins), hsp60, hsp70, hsp90, and hsp100, where the numbers reflect the approximate molecular weight of the hsps in kilodaltons. In addition to the major hsp families, an endoplasmic reticulum resident protein, calreticulin, has also been identified as yet another heat shock protein useful for eliciting an immune response when complexed to antigenic molecules (Basu and. Srivastava, 1999, J. Exp. Med. 189:797-202). Many members of these families were found subsequently to be induced in response to other stressful stimuli including, but not limited to, nutrient deprivation, metabolic disruption, oxygen radicals, and infection with intracellular pathogens. (See Welch, May 1993, Scientific American 56-64; Young, 1990, Annu. Rev. Immunol. 8:401-420; Craig, 1993, Science 260:1902-1903; Gething, et al., 1992, Nature 355:33-45; and Lindquist, et al., 1988, Annu. Rev. Genetics 22:631-677), the disclosures of which are incorporated herein by reference. It is contemplated that hsps/stress proteins belonging to all of these three families can be used in the practice of the instant invention.

The major hsps can accumulate to very high levels in stressed cells, but they occur at low to moderate levels in cells that have not been stressed. For example, the highly inducible mammalian hsp70is hardly detectable at normal temperatures but becomes one of the most actively synthesized proteins in the cell upon heat shock (Welch, et al., 1985, J. Cell. Biol. 101:1198-1211). In contrast, hsp90 and hsp60 proteins are abundant at normal temperatures in most, but not all, mammalian cells and are further induced by heat (Lai, et al., 1984, Mol. Cell. Biol. 4:2802-10; van Bergen en Henegouwen, et al., 1987, Genes Dev. 1:525-31).

Heat shock proteins are among the most highly conserved proteins in existence. For example, DnaK, the hsp70 from E. coli has about 50% amino acid sequence identity with hsp70proteins from excoriates (Bardwell, et al., 1984, Proc. Natl. Acad. Sci. 81:848-852). The hsp60 and hsp90families also show similarly high levels of intrafamilies conservation (Hickey, et al., 1989, Mol. Cell. Biol. 9:2615-2626; Jindal, 1989, Mol. Cell. Biol. 9:2279-2283). In addition, it has been discovered that the hsp60, hsp70 and hsp90families are composed of proteins that are related to the stress proteins in sequence, for example, having greater than 35% amino acid identity, but whose expression levels are not altered by stress. Therefore it is contemplated that the definition of heat shock protein or stress protein, as used herein, embraces other proteins, muteins, analogs, and variants thereof having at least 35% to 55%, preferably 55% to 75%, and most preferably 75% to 85% amino acid identity with members of the three families whose expression levels in a cell are enhanced in response to a stressful stimulus. The purification of exemplary hsp proteins is described below, as is the production of hsps by recombinant means.

4.2.1. Preparation and Purification of Hsp70-peptide Complexes

The purification of hsp70-peptide complexes has been described previously, see, for example, Udono et al., 1993, J Exp. Med. 178:1391-1396. A procedure that may be used, presented by way of example but not limitation, is as follows:

Initially, tumor cells are suspended in 3 volumes of 1×Lysis buffer consisting of 30 mM sodium bicarbonate pH 7.5, and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate pH 7.5, 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose equilibrated with phosphate buffered saline (PBS) containing 2 mM Ca²⁺ and 2 mM Mg²⁺. When the cells are lysed by mechanical shearing the supernatant is diluted with an equal volume of 2×lysis buffer prior to mixing with Con A Sepharose. The supernatant is then allowed to bind to the Con A Sepharose for 2-3 hours at 4° C. The material that fails to bind is harvested and dialyzed for 36 hours (three times, 100 volumes each time) against 10 mM Tris-Acetate pH 7.5, 0.1 mM EDTA, 10 mM NaCl, 1 mM PMSF. Then the dialyzate is centrifuged at 17,000 rpm (Sorvall SS34 rotor) for 20 minutes. Then the resulting supernatant is harvested and applied to a Mono Q FPLC column equilibrated in 20 mM Tris-Acetate pH 7.5, 20 mM NaCl, 0.1 mM EDTA and 15 mM 2-mercaptoethanol. The column is then developed with a 20 mM to 500 mM NaCl gradient and then eluted fractions fractionated by sodium dodecyl sulfate-polyacrylamide gel electrophoresis (SDS-PAGE) and characterized by immunoblotting using an appropriate anti-hsp70antibody (such as from clone N27F3-4, from StressGen).

Fractions strongly immunoreactive with the anti-hsp70 antibody are pooled and the hsp70-peptide complexes precipitated with ammonium sulfate; specifically with a 50%-70% ammonium sulfate cut. The resulting precipitate is then harvested by centrifugation at 17,000 rpm (SS34 Sorvall rotor) and washed with 70% ammonium sulfate. The washed precipitate is then solubilized and any residual ammonium sulfate removed by gel filtration on a Sephadex^{R} G25 column (Pharmacia). If necessary the hsp70preparation thus obtained can be repurified through the Mono Q FPLC Column as described above.

The hsp70-peptide complex can be purified to apparent homogeneity using this method. Typically 1 mg of hsp70-peptide complex can be purified from 1 g of cells/tissue.

An improved method for purification of hsp70-peptide complexes comprises contacting cellular proteins with ADP or a nonhydrolyzable analog of ATP affixed to a solid substrate, such that hsp70 in the lysate can bind to the ADP or nonhydrolyzable ATP analog, and eluting the bound hsp70. A preferred method uses column chromatography with ADP affixed to a solid substratum (e.g., ADP-agarose). The resulting hsp70preparations are higher in purity and devoid of contaminating peptides. The hsp70 yields are also increased significantly by about more than 10 fold. Alternatively, chromatography with nonhydrolyzable analogs of ATP, instead of ADP, can be used for purification of hsp70-peptide complexes. By way of example but not limitation, purification of hsp70-peptide complexes by ADP-agarose chromatography can be carried out as follows:

Meth A sarcoma cells (500 million cells) are homogenized in hypotonic buffer and the lysate is centrifuged at 100,000 g for 90 minutes at 4° C. The supernatant is applied to an ADP-agarose column. The column is washed in buffer and is eluted with 5 column volumes of 3 mM ADP. The hsp70-peptide complexes elute in fractions 2 through 10 of the total 15 fractions which elute. The eluted fractions are analyzed by SDS-PAGE. The hsp70-peptide complexes can be purified to apparent homogeneity using this procedure.

4.2.2. Preparation and Purification of Hsp90-peptide Complexes

A procedure that can be used, presented by way of example and not limitation, is as follows:

Initially, tumor cells are suspended in 3 volumes of 1×Lysis buffer consisting of 30 mM sodium bicarbonate pH 7.5, and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate pH 7.5, 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose equilibrated with PBS containing 2 mM Ca²⁺ and 2 mM Mg²⁺. When the cells are lysed by mechanical shearing the supernatant is diluted with an equal volume of 2×Lysis buffer prior to mixing with Con A Sepharose. The supernatant is then allowed to bind to the Con A Sepharose for 2-3 hours at 4° C. The material that fails to bind is harvested and dialyzed for 36 hours (three times, 100 volumes each time) against 10 mM Tris-Acetate pH 7.5, 0.1 mM EDTA, 10 mM NaCl, 1 mM PMSF. Then the dialyzate is centrifuged at 17,000 rpm (Sorvall SS34 rotor) for 20 minutes. Then the resulting supernatant is harvested and applied to a Mono Q FPLC column equilibrated with lysis buffer. The proteins are then eluted with a salt gradient of 200 mM to 600 mM NaCl.

The eluted fractions are fractionated by SDS-PAGE and fractions containing the hsp90-peptide complexes identified by immunoblotting using an anti-hsp90 antibody such as 3G3 (Affinity Bioreagents). hsp90-peptide complexes can be purified to apparent homogeneity using this procedure. Typically, 150-200 μg of hsp90-peptide complex can be purified from 1 g of cells/tissue.

4.2.3. Preparation and Purification of Gp96-peptide Complexes

A procedure that can be used, presented by way of example and not limitation, is as follows:

A pellet of tumors is resuspended in 3 volumes of buffer consisting of 30 mM sodium bicarbonate buffer (pH 7.5) and 1mM PMSF and the cells allowed to swell on ice 20 minutes. The cell pellet is then homogenized in a Dounce homogenizer (the appropriate clearance of the homogenizer will vary according to each cell type) on ice until >95% cells are lysed.

The lysate is centrifuged at 1,000g for 10 minutes to remove unbroken cells, nuclei and other debris. The supernatant from this centrifugation step is then recentrifuged at 100,000g for 90 minutes. The gp96-peptide complex can be purified either from the 100,000 pellet or from the supernatant.

When purified from the supernatant, the supernatant is diluted with equal volume of 2×lysis buffer and the supernatant mixed for 2-3 hours at 4° C. with Con A Sepharose equilibrated with PBS containing 2 mM Ca²⁺ and 2 mM Mg²⁺. Then, the slurry is packed into a column and washed with 1×lysis buffer until the OD₂₈₀ drops to baseline. Then, the column is washed with ⅓ column bed volume of 10% α-methyl mannoside (α-MM) dissolved in PBS containing 2 mM Ca²⁺ and 2 mM Mg²⁺, the column sealed with a piece of parafilm, and incubated at 37° C. for 15 minutes. Then the column is cooled to room temperature and the parafilm removed from the bottom of the column. Five column volumes of the α-MM buffer are applied to the column and the eluate analyzed by SDS-PAGE. Typically the resulting material is about 60-95% pure, however this depends upon the cell type and the tissue-to-lysis buffer ratio used. Then the sample is applied to a Mono Q FPLC column (Pharmacia) equilibrated with a buffer containing 5 mM sodium phosphate, pH 7. The proteins are then eluted from the column with a 0-1M NaCl gradient and the gp96 fraction elutes between 400 mM and 550 mM NaCl.

The procedure, however, may be modified by two additional steps, used either alone or in combination, to consistently produce apparently homogeneous gp96-peptide complexes. One optional step involves an ammonium sulfate precipitation prior to the Con A purification step and the other optional step involves DEAE-Sepharose purification after the Con A purification step but before the Mono Q FPLC step.

In the first optional step, described by way of example as follows, the supernatant resulting from the 100,000g centrifugation step is brought to a final concentration of 50% ammonium sulfate by the addition of ammonium sulfate. The ammonium sulfate is added slowly while gently stirring the solution in a beaker placed in a tray of ice water. The solution is stirred from about ½ to 12 hours at 4° C. and the resulting solution centrifuged at 6,000 rpm (Sorvall SS34 rotor). The supernatant resulting from this step is removed, brought to 70% ammonium sulfate saturation by the addition of ammonium sulfate solution, and centrifuged at 6,000 rpm (Sorvall SS34 rotor). The resulting pellet from this step is harvested and suspended in PBS containing 70% ammonium sulfate in order to rinse the pellet. This mixture is centrifuged at 6,000 rpm (Sorvall SS34 rotor) and the pellet dissolved in PBS containing 2 mM Ca²⁺ and Mg²⁺. Undissolved material is removed by a brief centrifugation at 15,000 rpm (Sorvall SS34 rotor). Then, the solution is mixed with Con A Sepharose and the procedure followed as before.

In the second optional step, described by way of example as follows, the gp96 containing fractions eluted from the Con A column are pooled and the buffer exchanged for 5 mM sodium phosphate buffer, pH 7, 300 mM NaCl by dialysis, or preferably by buffer exchange on a Sephadex G25 column. After buffer exchange, the solution is mixed with DEAE-Sepharose previously equilibrated with 5 mM sodium phosphate buffer, pH 7, 300 mM NaCl. The protein solution and the beads are mixed gently for 1 hour and poured into a column. Then, the column is washed with 5 mM sodium phosphate buffer, pH 7, 300 mM NaCl, until the absorbance at 280 nm drops to baseline. Then, the bound protein is eluted from the column with five volumes of 5 mM sodium phosphate buffer, pH 7, 700 mM NaCl. Protein containing fractions are pooled and diluted with 5 mM sodium phosphate buffer, pH 7 in order to lower the salt concentration to 175 mM. The resulting material then is applied to the Mono Q FPLC column (Pharmacia) equilibrated with 5 mM sodium phosphate buffer, pH 7 and the protein that binds to the Mono Q FPLC column (Pharmacia) is eluted as described before.

It is appreciated, however, that one skilled in the art may assess, by routine experimentation, the benefit of incorporating the second optional step into the purification protocol. In addition, it is appreciated also that the benefit of adding each of the optional steps will depend upon the source of the starting material.

When the gp96 fraction is isolated from the 100,000 g pellet, the pellet is suspended in 5 volumes of PBS containing either 1% sodium deoxycholate or 1% oxtyl glucopyranoside (but without the Mg²⁺ and Ca²⁺) and incubated on ice for 1 hour. The suspension is centrifuged at 20,000g for 30 minutes and the resulting supernatant dialyzed against several changes of PBS (also without the Mg²⁺ and Ca²⁺) to remove the detergent. The dialysate is centrifuged at 100,000 g for 90 minutes, the supernatant harvested, and calcium and magnesium are added to the supernatant to give final concentrations of 2 mM, respectively. Then the sample is purified by either the unmodified or the modified method for isolating gp96-peptide complex from the 100,000 g supernatant, see above.

The gp96-peptide complexes can be purified to apparent homogeneity using this procedure. About 10-20 μg of gp96 can be isolated from 1 g cells/tissue.

4.2.4. Preparation and Purification of Hsp110-peptide Complexes

A procedure, described by Wang et al., 2001, J. Immunol. 166(1):490-7, that can be used, presented by way of example and not limitation, is as follows:

A pellet (40-60 ml) of cell or tissue, e.g., tumor cell tissue, is homogenized in 5 vol of hypotonic buffer (30 mN sodium bicarbonate, pH7.2, and protease inhibitors) by Dounce homogenization. The lysate is centrifuged at 4,500×g and then 100,000×g for 2 hours. If the cells or tissues are of hepatic origin, the resulting supernatant is was first applied to a blue Sepharose column (Pharmacia) to remove albumin. Otherwise, the resulting supernatant is applied to a Con A-Sepharose column (Pharmacia Biotech, Piscataway, N.J.) previously equilibrated with binding buffer (20 mM Tris-HCl, pH 7.5; 100 mM NaCl; 1 mM MgCl₂; 1 mM CaCl₂; 1 mM MnCl₂; and 15 mM 2-ME). The bound proteins are eluted with binding buffer containing 15% α-D-o-methylmannoside (Sigma, St. Louis, Mo.).

Con A-Sepharose unbound material is first dialyzed against a solution of 20 mM Tris-HCl, pH 7.5; 100 mM NaCl; and 15 mM 2-ME, and then applied to a DEAE-Sepharose column and eluted by salt gradient from 100 to 500 mM NaCl. Fractions containing hsp 110 are collected, dialyzed, and loaded onto a Mono Q (Pharmacia) 10/10 column equilibrated with 20 mM Tris-HCl, pH 7.5; 200 mM NaCl; and 15 mM 2-ME. The bound proteins are eluted with a 200-500 mM NaCl gradient. Fractions are analyzed by SDS-PAGE followed by immunoblotting with an Ab for grp110, as described by Wang et al., 1999, J. Immunol. 162:3378. Pooled fractions containing hsp 110 are concentrated by Centriplus (Amicon, Beverly, Mass.) and applied to a Superose 12 column (Pharmacia). Proteins are eluted by 40 mM Tris-HCl, pH 8.0; 150 mM NaCl; and 15 mM 2-ME with a flow rate of 0.2 ml/min.

4.2.5. Preparation and Purification of Grp1170-peptide Complexes

A procedure, described by Wang et al., 2001, J. Immunol. 166(1):490-7, that can be used, presented by way of example and not limitation, is as follows:

A pellet (40-60 ml) of cell or tissue, e.g., tumor cell tissue, is homogenized in 5 vol of hypotonic buffer (30 mN sodium bicarbonate, pH7.2, and protease inhibitors) by 35 Dounce homogenization. The lysate is centrifuged at 4,500×g and then 100,000×g for 2 hours. If the cells or tissues are of hepatic origin, the resulting supernatant is was first applied to a blue Sepharose column (Pharmacia) to remove albumin. Otherwise, the resulting supernatant is applied to a Con A-Sepharose column (Pharmacia Biotech, Piscataway, N.J.) previously equilibrated with binding buffer (20 mM Tris-HCl, pH 7.5; 100 mM NaCl; 1 mM MgCl₂; 1 mM CaCl₂; 1 mM MnCl₂; and 15 mM 2-ME). The bound proteins are eluted with binding buffer containing 15% α-D-o-methylmannoside (Sigma, St. Louis, Mo.).

Con A-Sepharose-bound material is first dialyzed against 20 mM Tris-HCl, pH 7.5, and 150 mM NaCl and then applied to a Mono Q column and eluted by a 150 to 400 mM NaCl gradient. Pooled fractions are concentrated and applied on the Superose 12 column (Pharmacia). Fractions containing homogeneous grp 170 are collected.

4.2.6. α2M-Antigenic Molecule Complexes

Described below are methods for purifying α2M polypeptides or α2M polypeptide-antigenic molecule complexes for use in the invention from recombinant cells, and, with minor modifications known in the art, the α2M polypeptide or α2M-antigenic molecule complexes from cell culture. Recombinant cells include, for example, cells expressing antigenic molecules and recombinantly expressing an α2M polypeptide. Such cells may be derived from a variety of sources, including, but not limited to, cells infected with an infectious agent and cancer cells.

The invention provides methods for purification of recombinant α2M polypeptide-antigenic molecule complexes by affinity purification, based on the properties of the affinity label present on the α2M polypeptide. One approach is based on specific molecular interactions between a tag and its binding partner. The other approach relies on the immunospecific binding of an antibody to an epitope present on the tag. The principle of affinity chromatography well known in the art is generally applicable to both of these approaches.

To produce α2M polypeptide-antigenic molecule complexes, a nucleotide sequence encoding an α2M polypeptide can be introduced into a cell. When an antigenic molecule is present in the cell, the α2M polypeptide can associate intracellularly with the antigenic molecule, forming a covalent or a noncovalent complex of α2M polypeptide and the antigenic molecule. Cells into which an α2M polypeptide-encoding nucleotide sequence can be introduced, include, but are not limited to, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. The choice of cell type depends on the type of tumor or infectious disease being treated or prevented, and can be determined by one of skill in the art. In a specific embodiment, an expression construct comprising a nucleic acid sequence encoding the α2M polypeptide is introduced into an antigenic cell. As used herein, antigenic cells may include cells that are infected with an infectious agent or pathogen, cells infected with non-infectious or non-pathogenic forms of an infectious agent or pathogen (e.g., by use of a helper infectious agent), cells infected by or engineered to express an attenuated form of an infectious agent or a non-pathogenic or replication-deficient variant of a pathogen, pre-neoplastic cells that are infected with a cancer-causing infectious agent, such as a virus, but which are not yet neoplastic; or antigenic cells that have been exposed to a mutagen or cancer-causing agent, such as, for example DNA-damaging agents, radiation, etc. Other cells that can be used are pre-neoplastic cells which are in transition from a normal to a neoplastic form as characterized by morphology, physiological or biochemical functions. Preferably, the cancer cells and pre-neoplastic cells used in the methods of the invention are of mammalian origin. Mammals contemplated by this aspect of the invention include humans, companion animals (e.g., dogs and cats), livestock animals (e.g. sheep, cattle, goats, pigs and horses), laboratory animals (e.g., mice, rats and rabbits), and captive or free wild animals.

In various embodiments, any cancer cell, preferably a human cancer cell, can be used in the present methods for producing α2M polypeptide-antigenic molecule complexes. The cancer cells provide the antigenic peptides which become associated covalently or noncovalently with the expressed α2M polypeptide. α2M polypeptide-antigenic molecule complexes are then purified from the cells and used to treat such cancers. Cancers which can be treated or prevented with immunogenic compositions prepared by methods of the invention include, but are not limited to, tumors such as sarcomas and carcinomas. Examples of cancers that are amenable to the methods of the invention are listed in Section 4.9. Accordingly, any tissues or cells isolated from a pre-neoplastic lesion, a cancer, including cancer that has metastasized to multiple remote sites, can be used in the present method. For example, cells found in abnormally growing tissue, circulating leukemic cells, metastatic lesions as well as solid tumor tissue can be used.

In another embodiment, cell lines derived from a pre-neoplastic lesion, cancer tissues or cancer cells can also be used, provided that the cells of the cell line have at least one or more antigenic determinants in common with antigens on the target cancer cells. Cancer tissues, cancer cells, cells infected with a cancer-causing agent, other pre-neoplastic cells, and cell lines of human origin are preferred.

Cancer and pre-neoplastic cells can be identified by any method known in the art. For example, cancer cells can be identified by morphology, enzyme assays, proliferation assays, cytogenetic characterization, DNA mapping, DNA sequencing, the presence of cancer-causing virus, or a history of exposure to mutagen or cancer-causing agent, imaging, etc. Cancer cells may also be obtained by surgery, endoscopy, or other biopsy techniques. If some distinctive characteristics of the cancer cells are known, they can also be obtained or purified by any biochemical or immunological methods known in the art, such as but not limited to affinity chromatography, and fluorescence activated cell sorting (e.g., with fluorescently tagged antibody against an antigen expressed by the cancer cells).

Cancer tissues, cancer cells or cell lines may be obtained from a single individual or pooled from several individuals. It is not essential that clonal, homogeneous, or purified population of cancer cells be used. It is also not necessary to use cells of the ultimate target in vivo (e.g., cells from the tumor of the intended recipient), so long as at least one or more antigenic determinants on the target cancer cells is present on the cells used for expression of the α2M polypeptide. In addition, cells derived from distant metastases may be used to prepare an immunogenic composition against the primary cancer. A mixture of cells can be used provided that a substantial number of cells in the mixture are cancer cells and share at least one antigenic determinant with the target cancer cell. In a specific embodiment, the cancer cells to be used in expressing an α2M polypeptide are purified.

4.2.7. Preparation of Hsp Complexes for Treatment or Prevention of Infectious Disease

In an alternative embodiment wherein it is desired to treat a patient having an infectious disease, the above-described methods in Sections 4.2.1-4.2.5 are used to isolate hsp-peptide complexes from cells infected with an infectious organism or transfected with an expression construct of an antigen of an infectious agent, e.g., of a cell line or from a patient. The methods of Section 4.2.6 can be similarly used to isolate α2M-peptide complexes from cells that are infected with an infectious agent or cells that express antigens of infectious agents. Such infectious organisms include but are not limited to, viruses, bacteria, protozoa, fungi, and parasites as described in detail in Section 4.9.1 below.

4.3. Antigenic Molecules

The following subsections provide an overview of peptides that are useful as antigenic/immunogenic components of the Specific Complexes of the invention, and how such peptides can be identified, e.g., for use in recombinant expression of the peptides for in vitro complexing of hsps and antigenic molecules. However, in the practice of the present invention, the identity of the antigenic molecule(s) of the Specific Complex need not be known, for example when the Specific Complex is purified directly from a cancerous cell or from a tissue infected with a pathogen.

4.3.1. Isolation of Antigenic/Immunogenic Components

It has been found that antigenic peptides and/or components can be eluted from hsp-complexes either in the presence of ATP or low pH. These experimental conditions may be used to isolate peptides and/or antigenic components from cells which may contain potentially useful antigenic determinants. Once isolated, the amino acid sequence of each antigenic peptide may be determined using conventional amino acid sequencing methodologies. Such antigenic molecules can then be produced by chemical synthesis or recombinant methods, purified, and complexed to hsps in vitro to form the Specific Complexes of the invention.

Similarly, it has been found that potentially immunogenic peptides may be eluted from MHC-peptide complexes using techniques well known in the art (Falk, K. et al., 1990 Nature 348:248-251; Elliott, T., et al., 1990, Nature 348:195-197; Falk, K., et al., 1991, Nature 351:290-296).

Thus, potentially immunogenic or antigenic peptides may be isolated from either endogenous stress protein-peptide complexes or endogenous MHC-peptide complexes for use subsequently as antigenic molecules, by complexing in vitro to hsps to form the Specific Complexes of the invention. Exemplary protocols for isolating peptides and/or antigenic components from either of these complexes are set forth below in Sections 4.3.2 and 4.3.3.

4.3.2. Peptides From Stress Protein-peptide Complexes

Two methods may be used to elute the peptide from a stress protein-peptide complex. One approach involves incubating the stress protein-peptide complex in the presence of ATP. The other approach involves incubating the complexes in a low pH buffer.

Briefly, the complex of interest is centrifuged through a Centricon 10 assembly (Millipore) to remove any low molecular weight material loosely associated with the complex. The large molecular weight fraction may be removed and analyzed by SDS-PAGE while the low molecular weight may be analyzed by HPLC as described below. In the ATP incubation protocol, the stress protein-peptide complex in the large molecular weight fraction is incubated with 10 mM ATP for 30 minutes at room temperature. In the low pH protocol, acetic acid or trifluoroacetic acid (TFA) is added to the stress protein-peptide complex to give a final concentration of 10% (vol/vol) and the mixture incubated at room temperature or in a boiling water bath or any temperature in between, for 10 minutes See, Van Bleek, et al., 1990, Nature 348:213-216; and Li, et al., 1993, EMBO Journal 12:3143-3151).

The resulting samples are centrifuged through a Centricon 10 assembly as mentioned previously. The high and low molecular weight fractions are recovered. The remaining large molecular weight stress protein-peptide complexes can be reincubated with ATP or low pH to remove any remaining peptides.

The resulting lower molecular weight fractions are pooled, concentrated by evaporation and dissolved in 0.1% TFA. The dissolved material is then fractionated by reverse phase high pressure liquid chromatography (HPLC) using for example a VYDAC C18 reverse phase column equilibrated with 0.1% TFA. The bound material is then eluted at a flow rate of about 0.8 ml/min by developing the column with a linear gradient of 0 to 80% acetonitrile in 0.1% TFA. The elution of the peptides can be monitored by OD₂₁₀ and the fractions containing the peptides collected.

4.3.3. Peptides from MHC-peptide Complexes

The isolation of potentially immunogenic peptides from MHC molecules is well known in the art and so is not described in detail herein (See, Falk, et al., 1990, Nature 348:248-251; Rotzsche, at al., 1990, Nature 348:252-254; Elliott, et al., 1990, Nature 348:191-197; Falk, et al., 1991, Nature 351:290-296; Demotz, et al., 1989, Nature 343:682-684; Rotzsche, et al., 1990, Science 249:283-287), the disclosures of which are incorporated herein by reference.

Briefly, MHC-peptide complexes may be isolated by a conventional immunoaffinity procedure. The peptides then may be eluted from the MHC-peptide complex by incubating the complexes in the presence of about 0.1% TFA in acetonitrile. The eluted peptides may be fractionated and purified by reverse phase HPLC, as before.

The amino acid sequences of the eluted peptides may be determined either by manual or automated amino acid sequencing techniques well known in the art. Once the amino acid sequence of a potentially protective peptide has been determined the peptide may be synthesized in any desired amount using conventional peptide synthesis or other protocols well known in the art.

Peptides having the same amino acid sequence as those isolated above may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked y its C-terminal and to an insoluble polymeric support i.e., polystyrene beads. The eptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile.

Briefly, the C-terminal N-α-protected amino acid is first attached to the polystyrene beads. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting peptides is accomplished using conventional procedures, such as preparative HPLC using gel permeation, partition and/or ion exchange chromatography. The choice of appropriate matrices and buffers are well known in the art and so are not described in detail herein.

4.3.4. Exogenous Antigenic Molecules

Antigens or antigenic portions thereof can be selected for use as antigenic molecules, for complexing to hsps, from among those known in the art or determined by immunoassay to be able to bind to antibody or MHC molecules (antigenicity) or generate immune response (immunogenicity). To determine immunogenicity or antigenicity by detecting binding to antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in vivo immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are envisioned for use. In one embodiment for detecting immunogenicity, T cell-mediated responses can be assayed by standard methods, e.g., in vitro cytoxicity assays or in vivo delayed-type hypersensitivity assays.

Potentially useful antigens or derivatives thereof for use as antigenic molecules can also be identified by various criteria, such as the antigen's involvement in neutralization of a pathogen's infectivity (wherein it is desired to treat or prevent infection by such a pathogen) (Norrby, 1985, Summary, in Vaccines 85, Lerner, et al. (eds.), Cold Spring Harbor Laboratory, Cold Spring Harbor, N.Y., pp. 388-389), type or group specificity, recognition by patients' antisera or immune cells, and/or the demonstration of protective effects of antisera or immune cells specific for the antigen. In addition, where it is desired to treat or prevent a disease caused by pathogen, the antigen's encoded epitope should preferably display a small or no degree of antigenic variation in time or amongst different isolates of the same pathogen.

Preferably, where it is desired to treat or prevent cancer, known tumor-specific antigens or fragments or derivatives thereof are used. For example, such tumor specific or tumor-associated antigens include but are not limited to KS ¼ pan-carcinoma antigen (Perez and Walker, 1990, J. Immunol. 142:3662-3667; Bumal, 1988, Hybridoma 7(4):407-415); ovarian carcinoma antigen (CA125) (Yu, et al., 1991, Cancer Res. 51(2):468-475); prostatic acid phosphate (Tailer, et al., 1990, Nucl. Acids Res. 18(16):4928); prostate specific antigen (Henttu and Vihko, 1989, Biochem. Biophys. Res. Comm. 160(2):903-910; Israeli, et al., 1993, Cancer Res. 53:227-230); melanoma associated antigen p97 (Estin, et al., 1989, J. Natl. Cancer Inst. 81(6):445-446); melanoma antigen gp75 (Vijayasardahl, et al., 1990, J. Exp. Med. 171(4):1375-1380); high molecular weight melanoma antigen (Natali, et al., 1987, Cancer 59:55-63) and prostate specific membrane antigen. Other exogenous antigens that may be complexed to hsps include portions or proteins that are mutated at a high frequency in cancer cells, such as oncogenes (e.g., ras, in particular mutants of ras with activating mutations, which only occur in four amino acid residues (12, 13, 59 or 61) (Gedde-Dahl et al., 1994, Eur. J. Immunol. 24(2):410-414)) and tumor suppressor genes (e.g., p53, for which a variety of mutant or polymorphic p53 peptide antigens capable of stimulating a cytotoxic T cell response have been identified (Gnjatic et al., 1995, Eur. J. Immunol. 25(6):1638-1642).

In a specific embodiment, an antigen or fragment or derivative thereof specific to a certain tumor is selected for complexing to hsp to form a Specific Complex and subsequently mixed with a Diluent for administration to a patient having that tumor.

Preferably, where it is desired to treat or prevent viral diseases, molecules comprising epitopes of known viruses are used. For example, such antigenic epitopes may be prepared from viruses including, but not limited to, hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II). Preferably, where it is desired to treat or prevent bacterial infections, molecules comprising epitopes of known bacteria are used. For example, such antigenic epitopes may be prepared from bacteria including, but not limited to, mycobacteria rickettsia, mycoplasma, neisseria and legionella.

Preferably, where it is desired to treat or prevent protozoal infections, molecules comprising epitopes of known protozoa are used. For example, such antigenic epitopes may be prepared from protozoa including, but not limited to, leishmania, kokzidioa, and trypanosoma.

Preferably, where it is desired to treat or prevent parasitic infections, molecules comprising epitopes of known parasites are used. For example, such antigenic epitopes may be from parasites including, but not limited to, chlamydia and rickettsia.

4.4. In Vitro Production of Stress Protein-antigenic Molecule Complexes

In an embodiment in which Specific Complexes of hsps or α2M and the peptides with which they are endogenously associated in vivo are not employed, complexes of hsps to antigenic molecules are produced in vitro. As will be appreciated by those skilled in the art, the peptides either isolated by the aforementioned procedures or chemically synthesized or recombinantly produced may be reconstituted with a variety of purified natural or recombinant stress proteins in vitro to generate immunogenic non-covalent stress protein-antigenic molecule complexes. Alternatively, exogenous antigens or antigenic or immunogenic fragments or derivatives thereof can be complexed to stress proteins for use as the Specific Complexes of the immunotherapeutic or prophylactic vaccines of the invention. A preferred, exemplary protocol for complexing a stress protein and an antigenic molecule in vitro is discussed below.

Prior to complexing, the hsps are pretreated with ATP or low pH to remove any peptides that may be associated with the hsp of interest. When the ATP procedure is used, excess ATP is removed from the preparation by the addition of apyranase as described by Levy, et al., 1991, Cell 67:265-274. When the low pH procedure is used, the buffer is readjusted to neutral pH by the addition of pH modifying reagents.

The antigenic molecules (1 μg) and the pretreated hsp (9 μg) are admixed to give an approximately 5 antigenic molecule: 1 stress protein molar ratio. Then, the mixture is incubated for 15 minutes to 3 hours at 4° to 45 ° C. in a suitable binding buffer such as one containing 20 mM sodium phosphate, pH 7.2, 350 mM NaCl, 3 mM MgCl₂ and 1 mM phenyl methyl sulfonyl fluoride (PMSF). The preparations are centrifuged through a Centricon 10 assembly (Millipore) to remove any unbound peptide. The association of the peptides with the stress proteins can be assayed by SDS-PAGE. This is the preferred method for in vitro complexing of peptides isolated from MHC-peptide complexes of peptides disassociated from endogenous hsp-peptide complexes.

In an alternative embodiment of the invention, preferred for producing complexes of hsp70 to exogenous antigenic molecules such as proteins, 5-10 micrograms of purified hsp is incubated with equimolar quantities of the antigenic molecule in 20 mM sodium phosphate buffer pH 7.5, 0.5M NaCl, 3 mM MgCl₂ and 1 mM ADP in a volume of 100 microliter at 37° C. for 1 hr. This incubation mixture is further diluted to 1 ml in phosphate-buffered saline.

In an alternative embodiment of the invention, preferred for producing complexes of gp96 or hsp90 to peptides, 5-10 micrograms of purified gp96 or hsp90 is incubated with equimolar or excess quantities of the antigenic peptide in a suitable buffer such as one containing 20 mM sodium phosphate buffer pH 7.5, 0.5 M NaCl, 3 nM MgCl2 at 60-65° C. for 5-20 min. This incubation mixture is allowed to cool to room temperature and centrifuged one or more times if necessary, through a Centricon 10 assembly (Millipore) to remove any unbound peptide.

Following complexing, an immunogenic stress protein-antigenic molecule complex can optionally be assayed in vitro using for example the mixed lymphocyte target cell assay (MLTC) described below. This assay can be carried out prior to or following mixing with a Diluent. Once Specific Complexes have been isolated and diluted, they can be optionally characterized further in animal models using the preferred administration protocols and excipients discussed below.

4.4.1. In Vitro Complexing of α2M and Antigenic Molecules

Complexes of α2M polypeptides and antigenic molecules may be produced in vitro. α2M polypeptide-antigenic molecule complexes can be generated in vitro by coupling of an α2M polypeptide with an antigenic peptide. Procedures for forming such α2M-antigenic molecule complexes are described below.

In general, when an α2M is mixed with a protease, cleavage of the “bait”region of α2M takes place, the proteinase becomes “trapped” by thioesters, and a conformational change takes place that allows binding of the α2M complex to the α2M receptor. During proteolytic activation of (α2M, non-proteolytic ligands can become covalently bound to the activated thioesters. Non-proteolytic ligands can also be incorporated into the activated α2M molecule by ammonia or methylamine during reversal of the nucleophilic activation, employing heat (Grøn and Pizzo, 1998, Biochemistry, 37: 6009-6014). Such conditions that allow fortuitous trapping of peptides by α2M are employed to prepare the α2M -antigenic complexes for use in the invention. Methods for such covalent coupling have been described previously (Osada et al., 1987, Biochem. Biophys. Res. Commun.146:26-31; Osada et al., 1988, Biochem. Biophys. Res. Commun. 150:883; Chu and Pizzo, 1993, J. Immunol. 150:48; Chu et al., 1994, Ann. N.Y. Acad. Sci. 737:291-307; Mitsuda et al., 1993, Biochem. Biophys. Res. Commun. 101:1326-1331). Thus in one embodiment, an α2M antigenic molecule complex can be prepared as described by Grøn and Pizzo, 1998, Biochemistry, 37: 6009-6014. The method of Grøn and Pizzo yields complexes of α2M that are covalently bound to antigenic molecules.

For example, α2M polypeptide is mixed with an antigenic molecule in the presence of a protease, ammonia or other small amine nucleophiles such as methylamine and ethylamine. Non-limiting examples of proteases which may be used include trypsin, porcine pancreatic elastase (PEP), human neutrophil elastase, cathepsin G, S. aureus V-8 proteinase trypsin, α-chymotrypsin, V8 protease, papain, and proteinase K (see Ausubel et al., eds., in “Current Protocols in Molecular Biology”, Greene Publishing Associates and Wiley Interscience, New York, 17.4.6-17.4.8). A preferred, exemplary protocol for complexing an α2M polypeptide and an antigenic molecule in vitro follows. The antigenic molecules (1 μg -20 mg) and the α2M polypeptide (1 μg-20 mg) are mixed together in phosphate-buffered saline (PBS) (100 μl-5 ml) in the presence of a protease, such as trypsin (0.92 mg trypsin in approximately 500 μl PBS, to give an approximately 5:1 antigenic molecule: α2M polypeptide molar ratio. The mixture is then incubated for 5-15 minutes at 37° C. 500 μl 4 mg/ml p-Aphenyl methyl sulfonyl fluoride (p-APMSF) is added to the solution to inhibit trypsin activity and incubated for 2 hrs at 25° C. The preparations can be centrifuged through a Centricon 10 assembly (Millipore) to remove any unbound peptide. Alternatively, free antigenic molecule may be removed by passage over a gel permeation column. The association of the peptides with the α2M polypeptide can be assayed by SDS-PAGE. This is the preferred method for in vitro complexing of antigenic molecules isolated from MIC-antigenic molecule complexes, or peptides disassociated from endogenous α2M-antigenic molecule complexes.

The foregoing methods of producing α2M-antigenic molecule complexes produce complexes in which the α2M polypeptide is covalently bound to the antigenic molecules. Covalent complexes of α2M and antigenic molecules can also be produced by the cross-linking methods described for heat shock proteins and antigenic molecules in Section 4.5, infra.

In a more preferred method, which produces non-covalent α2M-antigenic molecule complexes, an α2M-antigenic molecule complex is prepared according to the method described by Blachere et al., J. Exp. Med. 186(8):1315-22, which incorporated by reference herein in its entirety. Blachere teaches in vitro complexing of hsps to antigenic molecule. The protocol described in Blachere can be modified such that the hsp component is substituted by α2M. Binder et al. (2001, J. Immunol. 166:4968-72) demonstrates that the Blachere method yields complexes of α2M bound to antigenic molecules.

Antigenic molecules may be isolated from various sources, chemically synthesized, or produced recombinantly. Such methods can be readily adapted for medium or large scale production of the immunotherapeutic or prophylactic vaccines.

Following complexing, the immunogenic α2M-antigenic molecule complexes can optionally be assayed in vitro using, for example, the mixed lymphocyte target cell assay (MLTC) described below. Once immunogenic complexes have been isolated they can be optionally characterized further in animal models using the preferred administration protocols and excipients discussed below.

4.5. Formation of Covalent Hsp-peptide Complexes

As an alternative to non-covalent complexes of hsps or α2M and antigenic molecules, antigenic molecules may be covalently attached to hsps and/or α2M in either or both the Specific Complexes and Diluents prior to mixing or after the Specific Complexes and Diluents are mixed. Hsp-peptide complexes are preferably cross-linked after their purification from cells or tissues as described in Sections 4.2.1 to 4.2.5. Covalently linked complexes are the complexes of choice when a B cell response is desired. Methods of producing covalent (α2M-antigenic molecule complexes are described in § 4.2.6, supra.

In one embodiment, hsps are covalently coupled to antigenic molecules by chemical crosslinking. Chemical crosslinking methods are well known in the art. For example, in a preferred embodiment, glutaraldehyde crosslinking may be used. Glutaradehyde crosslinking has been used for formation of covalent complexes of peptides and hsps (see Barrios et al., 1992, Eur. J. Immunol. 22: 1365-1372). Preferably, 1-2 mg of hsp-peptide complex is crosslinked in the presence of 0.002% glutaraldehyde for 2 hours. Glutaraldehyde is removed by dialysis against phosphate buffered saline (PBS) overnight (Lussow et al., 1991, Eur. J. Immunol. 21: 2297-2302). In one embodiment, the following protocol is used. Optionally, hsps may be pretreated with ATP or low pH prior to complexing, in order to remove any peptides that may be associated with the hsps polypeptide. Preferably, 1 mg of hsp is crosslinked to 1 mg of peptide in the presence of 0.002% glutaraldehyde for 2 hours. Glutaraldehyde is removed by dialysis against phosphate buffered saline (PBS) overnight (Lussow et al., 1991, Eur. J. Immunol. 21: 2297-2302).

Other methods for chemical crosslinking may also be used, in addition other methods for covalent attachment of proteins, such as photocrosslinking (see Current Protocols in Molecular Biology, Ausubel et al. (eds.), Greene Publishing Associates and Wiley Interscience, New York).

In another embodiment, the hsp and specific antigen(s) are crosslinked by ultraviolet (UV) crosslinking.

4.6. α2M- or Hsp-antigenic Molecule Fusion Proteins

In certain embodiments of the invention, an α2M- or hsp-antigenic molecule complex is a recombinant fusion protein. Such recombinant fusion proteins, comprised of hsp or α2M sequences linked to antigenic molecule sequences, may be used in the Specific Complexes and/or the Diluents of the present invention. To produce such a recombinant fusion protein, an expression vector is constructed using nucleic acid sequences encoding the hsp or α2M fused to sequences encoding an antigenic molecule, using recombinant methods known in the art, such as those described in Section 4.7 below (see Suzue et al., 1997, Proc. Natl. Acad. Sci. U.S.A. 94: 13146-51). hsp- and α2M-antigenic peptide fusions are then expressed and isolated. By specifically designing the antigenic peptide portion of the molecule, such fusion proteins can be used to elicit an immune response and in immunotherapy against target cancer and infectious diseases.

4.7. Recombinant Expression of Hsps, α2M. and Antigenic Peptides

In certain embodiments of the invention, the compositions and methods comprise recombinant hsps, alone or complexed to antigenic molecules, or hsp-antigenic molecule complexes prepared from cells that express enhanced levels of hsps through recombinant means. In other embodiments of the invention, the compositions and methods comprise recombinant α2M or α2M-antigenic molecule complexes comprising recombinant α2M. In this regard, any method known to the skilled artisan may be used for obtaining and manipulating recombinant hsp or α2M sequences. Described below are non-limiting examples of such methods for recombinant expression of hsps or α2M. Such methods are also applicable for recombinant expression of antigenic molecules.

4.7.1. hsp Sequences

Amino acid sequences and nucleotide sequences of many hsps are generally available in sequence databases, such as GenBank. Computer programs, such as Entrez, can be used to browse the database, and retrieve any amino acid sequence and genetic sequence data of interest by accession number. These databases can also be searched to identify sequences with various degrees of similarities to a query sequence using programs, such as FASTA and BLAST, which rank the similar sequences by alignment scores and statistics. Such nucleotide sequences of non-limiting examples of hsps that can be used for the compositions, methods, and for preparation of the hsp-antigenic molecule complexes of the invention are as follows: human hsp70, Genbank Accession No.M24743, Hunt et al., 1995, Proc. Natl. Acad. Sci. U.S.A., 82: 6455-6489; human hsp90, Genbank Accession No.X15183, Yamazaki et al., Nucl. Acids Res. 17: 7108; human gp96, Genbank Accession No.X15187, Maki et al., 1990, Proc. Natl. Acad. Sci. U.S.A. 87: 5658-5562; human BiP, Genbank Accession No.M19645, Ting et al., 1988, DNA 7: 275-286; human hsp27, Genbank Accession No.M24743, Hickey et al., 1986, Nucleic Acids Res. 14: 4127-45; mouse hsp70, Genbank Accession No.M35021, Hunt et al, 1990, Gene 87: 199-204; mouse gp96, Genbank Accession No.M16370, Srivastava et al., 1987, Proc. Natl. Acad. Sci. U.S.A. 85: 3807-3811; and mouse BiP, Genbank Accession No.U16277, Haas et al., 1988, Proc. Natl. Acad. Sci. U.S.A. 85: 2250-2254. Due to the degeneracy of the genetic code, the term “hsp gene”, as used herein, refers not only to the naturally occurring nucleotide sequence but also encompasses all the other degenerate DNA sequences that encode the hsp.

Once the nucleotide sequence encoding the hsp of choice has been identified, the nucleotide sequence, or a fragment thereof, can be obtained (e.g. from commercial sources or by PCR as described below) and cloned into an expression vector for recombinant expression. The expression vector can then be introduced into a host cell for propogation of the hsp. Methods for recombinant production of hsps are quite well known, as exemplified herein.

The DNA may be obtained by DNA amplification or molecular cloning directly from a tissue, cell culture, or cloned DNA (e.g., a DNA “library”) using standard molecular biology techniques (see e.g., Methods in Enzymology, 1987, volume 154, Academic Press; Sambrook et al. 1989, Molecular Cloning—A Laboratory Manual, 2nd Edition, Cold Spring Harbor Press, New York; and Current Protocols in Molecular Biology, Ausubel et al. (eds.), Greene Publishing Associates and Wiley Interscience, New York, each of which is incorporated herein by reference in its entirety). Clones derived from genomic DNA may contain regulatory and intron DNA regions in addition to coding regions; clones derived from cDNA will contain only exon sequences. Whatever the source, the hsp gene should be cloned into a suitable vector for propagation of the gene.

In a preferred embodiment, DNA can be amplified from genomic or cDNA by polymerase chain reaction (PCR) amplification using primers designed from the known sequence of a related or homologous hsp. PCR is used to amplify the desired sequence in DNA clone or a genomic or cDNA library, prior to selection. PCR can be carried out, e.g., by use of a thermal cycler and Taq polymerase (Gene Amp®). The polymerase chain reaction (PCR) is commonly used for obtaining genes or gene fragments of interest. For example, a nucleotide sequence encoding an hsp of any desired length can be generated using PCR primers that flank the nucleotide sequence encoding open reading frame. Alternatively, an hsp gene sequence can be cleaved at appropriate sites with restriction endonuclease(s) if such sites are available, releasing a fragment of DNA encoding the hsp gene. If convenient restriction sites are not available, they may be created in the appropriate positions by site-directed mutagenesis and/or DNA amplification methods known in the art (see, for example, Shankarappa et al., 1992, PCR Method Appl. 1: 277-278). The DNA fragment that encodes the hsp is then isolated, and ligated into an appropriate expression vector, care being taken to ensure that the proper translation reading frame is maintained.

In an alternative embodiment, for the molecular cloning of an hsp gene from genomic DNA, DNA fragments are generated to form a genomic library. Since some of the sequences encoding related hsps are available and can be purified and labeled, the cloned DNA fragments in the genomic DNA library may be screened by nucleic acid hybridization to a labeled probe (Benton and Davis, 1977, Science 196: 180; Grunstein and Hogness, 1975, Proc. Natl. Acad. Sci. U.S.A. 72: 3961). Those DNA fragments with substantial homology to the probe will hybridize. It is also possible to identify an appropriate fragment by restriction enzyme digestion(s) and comparison of fragment sizes with those expected according to a known restriction map.

Alternatives to isolating the hsp genomic DNA include, but are not limited to, chemically synthesizing the gene sequence itself from a known sequence or synthesizing a cDNA to the mRNA which encodes the hsp. For example, RNA for cDNA cloning of the hsp gene can be isolated from cells which express the hsp. A cDNA library may be generated by methods known in the art and screened by methods, such as those disclosed for screening a genomic DNA library. If an antibody to the hsp is available, the hsp may be identified by binding of a labeled antibody to the hsp-synthesizing clones.

Other specific embodiments for the cloning of a nucleotide sequence encoding an hsp, are presented as examples but not by way of limitation, as follows: In a specific embodiment, nucleotide sequences encoding an hsp can be identified and obtained by hybridization with a probe comprising a nucleotide sequence encoding hsp under conditions of low to medium stringency. By way of example and not limitation, procedures using such conditions of low stringency are as follows (see also Shilo and Weinberg, 1981, Proc. Natl. Acad. Sci. U.S.A. 78: 6789-6792). Filters containing DNA are pretreated for 6 h at 40° C. in a solution containing 35% formamide, 5×SSC, 50 mM Tris-HCl (pH7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH7.4), SmM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson et al., 1978, J. Biol. Chem. 253: 6551), oligonucleotide-directed mutagenesis (Smith, 1985, Ann. Rev. Genet. 19: 423-463; Hill et al., 1987, Methods Enzymol. 155: 558-568), PCR-based overlap extension (Ho et al., 1989, Gene 77: 51-59), PCR-based megaprimer mutagenesis (Sarkar et al., 1990, Biotechniques 8: 404-407), etc. Modifications can be confirmed, e.g., by double-stranded dideoxynucleotide DNA sequencing.

In certain embodiments, a nucleic acid encoding a secretory form of the hsp of choice is used to prepare the compositions and/or practice the methods of the present invention. Such a nucleic acid can be constructed by, e.g., deleting the coding sequence for an ER retention signal, KDEL. Optionally, the KDEL coding sequence is replaced with a molecular tag, such as the Fc portion of murine IgG1, to facilitate the recognition and purification of the hsp. U.S. application Ser. No. 09/253,439, incorporated herein by reference, demonstrates that deletion of the ER retention signal of gp96 results in the secretion of gp96-Ig peptide-complexes from transfected tumor cells, and that fusion of the KDEL-deleted gp96 with murine IgG1 facilitated its detection by ELISA and FACS analysis, and its purification by affinity chromatography with the aid of Protein A.

4.7.2. α2M Sequences

α2M polypeptides may be produced by recombinant DNA techniques, synthetic methods, or by enzymatic or chemical cleavage of native α2M polypeptides. Described below are methods for producing such α2M polypeptides.

In various aspects, the invention relates to compositions comprising amino acid sequences of α2M, and fragments, derivatives, analogs, and variants thereof. Nucleic acids encoding α2M are provided, as well as nucleic acids complementary to and capable of hybridizing to such nucleic acids.

Any eukaryotic cell may serve as the nucleic acid source for obtaining the coding region of an α2M gene. Nucleic acid sequences encoding α2M can be isolated from vertebrate, mammalian, as well as primate sources, including humans.

Amino acid sequences and nucleotide sequences of naturally occurring α2M polypeptides are generally available in sequence databases, such as GenBank. Non-limiting examples of α2M sequences that can be used for preparation of the α2M polypeptides of the invention are as follows: Genbank AccessionNos. M11313, P01023, AAA51551; see Kan et al., 1985, Proc. Nat. Acad. Sci. U.S.A. 82: 2282-2286. Due to the degeneracy of the genetic code, the term “α2M gene”, as used herein, refers not only to the naturally occurring nucleotide sequence but also encompasses all the other degenerate DNA sequences that encode an α2M polypeptide. Computer programs, such as Entrez, can be used to browse the database, and retrieve any amino acid sequence and genetic sequence data of interest by accession number. These databases can also be searched to identify sequences with various degrees of similarities to a query sequence using programs, such as FASTA and BLAST, which rank the similar sequences by alignment scores and statistics. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res.25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov).

The DNA may be obtained by standard procedures known in the art by DNA amplification or molecular cloning directly from a tissue, cell culture, or cloned DNA (e.g., a DNA “library”). Clones derived from genomic DNA may contain regulatory and intron DNA regions in addition to coding regions; clones derived from cDNA will contain only exon sequences. Whatever the source, the α2M gene should be cloned into a suitable vector for propagation of the gene.

In a preferred embodiment, DNA can be amplified from genomic or cDNA by polymerase chain reaction (PCR) amplification using primers designed from the known sequence of a related or homologous α2M. PCR is used to amplify the desired sequence in DNA clone or a genomic or cDNA library, prior to selection. PCR can be carried out, e.g., by use of a thermal cycler and Taq polymerase (sold under the trademark GENE AMP). The DNA being amplified can include cDNA or genomic DNA from any species. Oligonucleotide primers representing known nucleic acid sequences of related HSPs can be used as primers in PCR. In a preferred aspect, the oligonucleotide primers represent at least part of the α2M gene that is highly conserved between α2M genes of different species. One can choose to synthesize several different degenerate primers, for use in the PCR reactions. It is also possible to vary the stringency of hybridization conditions used in priming the PCR reactions, to allow for greater or lesser degrees of nucleotide sequence similarity between the known α2M nucleotide sequence and the nucleic acid homolog being isolated. For cross species hybridization, low stringency conditions are preferred. For same species hybridization, moderately stringent conditions are preferred. After successful amplification, the sequence encoding an α2M may be cloned and sequenced. If the size of the coding region of the α2M gene being amplified is too large to be amplified in a single PCR, several PCR covering the entire gene, preferably with overlapping regions, may be carried out, and the products of the PCR ligated together to form the entire coding sequence. Alternatively, if a segment of an α2M gene is amplified, that segment may be cloned, and utilized as a probe to isolate a complete cDNA or genomic clone.

In another embodiment, for the molecular cloning of an α2M gene from genomic DNA, DNA fragments are generated to form a genomic library. Since some of the sequences encoding related α2Ms are available and can be purified and labeled, the cloned DNA fragments in the genomic DNA library may be screened by nucleic acid hybridization to the labeled probe (Benton and Davis, 1977, Science 196:180; Grunstein and Hogness, 1975, Proc. Natl. Acad. Sci. U.S.A. 72:3961). Those DNA fragments with substantial homology to the probe will hybridize. It is also possible to identify the appropriate fragment by restriction enzyme digestion(s) and comparison of fragment sizes with those expected according to a known restriction map if such is available.

Alternatives to isolating the α2M genomic DNA include, but are not limited to, chemically synthesizing the gene sequence itself from a known sequence or making cDNA to the mRNA which encodes α2M. For example, RNA for cDNA cloning of the α2M gene can be isolated from cells which express α2M. A cDNA library may be generated by methods known in the art and screened by methods, such as those disclosed for screening a genomic DNA library. If an antibody to α2M is available, α2M may be identified by binding of labeled antibody to the putatively α2M synthesizing clones.

Other specific embodiments for the cloning of a nucleotide sequence encoding an α2M, are presented as examples but not by way of limitation, as follows:

In a specific embodiment, nucleotide sequences encoding α2M proteins within a family can be identified and obtained by hybridization with a probe comprising nucleotide sequence encoding α2M under conditions of low to medium stringency.

By way of example and not limitation, procedures using such conditions of low stringency are as follows (see also Shilo and Weinberg, 1981, Proc. Natl. Acad. Sci. USA 78:6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

An α2M gene fragment can be inserted into an appropriate cloning vector and introduced into host cells so that many copies of the gene sequence are generated. A large number of vector-host systems known in the art may be used such as, but not limited to, bacteriophages such as lambda derivatives, or plasmids such as pBR322 or pUC plasmid derivatives or the Bluescript vector (Stratagene).

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson et al., 1978, J. Biol. Chem 253:6551), oligonucleotide-directed mutagenesis (Smith, 1985, Ann. Rev. Genet. 19:423-463; Hill et al., 1987, Methods Enzymol. 155:558-568), PCR-based overlap extension (Ho et al., 1989, Gene 77:51-59), PCR-based megaprimer mutagenesis (Sarkar et al., 1990, Biotechniques, 8:404-407), etc. Modifications can be confirmed by double stranded dideoxy DNA sequencing.

The polymerase chain reaction (PCR) is commonly used for obtaining genes or gene fragments of interest. For example, a nucleotide sequence encoding α2M polypeptide of any desired length can be generated using PCR primers that flank the nucleotide sequence encoding α2M, or the peptide-binding domain thereof. Alternatively, an α2M gene sequence can be cleaved at appropriate sites with restriction endonuclease(s) if such sites are available, releasing a fragment of DNA encoding α2M, or the peptide-binding domain thereof. If convenient restriction sites are not available, they may be created in the appropriate positions by site-directed mutagenesis and/or DNA amplification methods known in the art (see, for example, Shankarappa et al., 1992, PCR Method Appl. 1:277-278). The DNA fragment that encodes α2M, or the peptide-binding domain thereof, is then isolated, and ligated into an appropriate expression vector, care being taken to ensure that the proper translation reading frame is maintained.

Alpha (2) macroglobulin polypeptides may be expressed as fusion proteins to facilitate recovery and purification from the cells in which they are expressed. For example, an α2M polypeptide may contain a signal sequence leader peptide to direct its translocation across the ER membrane for secretion into culture medium. Further, an α2M polypeptide may contain an affinity label, such as a affinity label, fused to any portion of the α2M polypeptide not involved in binding antigenic peptide, such as for example, the carboxyl terminal. The affinity label can be used to facilitate purification of the protein, by binding to an affinity partner molecule.

Various methods for production of such fusion proteins are well known in the art. The manipulations which result in their production can occur at the gene or protein level, preferably at the gene level. For example, the cloned coding region of an α2M polypeptide may be modified by any of numerous recombinant DNA methods known in the art (Sambrook et al., 1990, Molecular Cloning, A Laboratory Manual, 2d ed., Cold Spring Harbor Laboratory, Cold Spring Harbor, New York; Ausubel et al., in Chapter 8 of Current Protocols in Molecular Biology, Greene Publishing Associates and Wiley Interscience, New York). It will be apparent from the following discussion that substitutions, deletions, insertions, or any combination thereof are introduced or combined to arrive at a final nucleotide sequence encoding an α2M polypeptide.

In various embodiments, fusion proteins comprising the α2M polypeptide may be made using recombinant DNA techniques. For example, a recombinant gene encoding an α2M polypeptide may be constructed by introducing an α2M gene fragment in the proper reading frame into a vector containing the sequence of an affinity label, such that the α2M polypeptide is expressed as a peptide-tagged fusion protein. Affinity labels, which may be recognized by specific binding partners, may be used for affinity purification of the α2M polypeptide.

In a preferred embodiment, the affinity label is fused at its amino terminal to the carboxyl terminal of α2M. The precise site at which the fusion is made in the carboxyl terminal is not critical. The optimal site can be determined by routine experimentation.

A variety of affinity labels known in the art may be used, such as, but not limited to, the immunoglobulin constant regions, polyhistidine sequence (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels may impart fluorescent properties to an α2M polypeptide, e.g., portions of green fluorescent protein and the like. Other possible affinity labels are short amino acid sequences to which monoclonal antibodies are available, such as but not limited to the following well known examples, the FLAG epitope, the myc epitope at amino acids 408-439, the influenza virus hemagglutinin (HA) epitope. Other affinity labels are recognized by specific binding partners and thus facilitate isolation by affinity binding to the binding partner which can be immobilized onto a solid support. Some affinity labels may afford the α2M polypeptide novel structural properties, such as the ability to form multimers. Dimerization of an α2M polypeptide with a bound peptide may increase avidity of interaction between the α2M polypeptide and its partner in the course of antigen presentation. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or portions of the immunoglobulin molecule containing sites for interchain disulfide bonds, could lead to the formation of multimers. As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

A preferred affinity label is a non-variable portion of the immunoglobulin molecule. Typically, such portions comprise at least a functionally operative CH2 and CH3 domain of the constant region of an immunoglobulin heavy chain. Fusions are also made using the carboxyl terminus of the Fc portion of a constant domain, or a region immediately amino-terminal to the CH1 of the heavy or light chain. Suitable immunoglobulin-based affinity label may be obtained from IgG-1, -2, -3, or -4 subtypes, IgA, IgE, IgD, or IgM, but preferably IgG1. Preferably, a human immunoglobulin is used when the α2M polypeptide is intended for in vivo use for humans. Many DNA encoding immunoglobulin light or heavy chain constant regions is known or readily available from cDNA libraries. See, for example, Adams et al., Biochemistry, 1980, 19:2711-2719; Gough et al., 1980, Biochemistry, 19:2702-2710; Dolby et al., 1980, Proc. Natl. Acad. Sci. U.S.A., 77:6027-6031; Rice et al., 1982, Proc. Natl. Acad. Sci. U.S.A., 79:7862-7865; Falkner et al., 1982, Nature, 298:286-288; and Morrison et al., 1984, Ann. Rev. Immunol, 2:239-256. Because many immunological reagents and labeling systems are available for the detection of immunoglobulins, the α2M polypeptide-Ig fusion protein can readily be detected and quantified by a variety of immunological techniques known in the art, such as the use of enzyme-linked immunosorbent assay (ELISA), immunoprecipitation, fluorescence activated cell sorting (FACS), etc. Similarly, if the affinity label is an epitope with readily available antibodies, such reagents can be used with the techniques mentioned above to detect, quantitate, and isolate the α2M polypeptide containing the affinity label. In many instances, there is no need to develop specific antibodies to the α2M polypeptide.

A particularly preferred embodiment is a fusion of an α2M polypeptide to the hinge, the CH2 and CH3 domains of human immunoglobulin G-1 (IgG-1; see Bowen et al.,1996, J. Immunol. 156:442-49). This hinge region contains three cysteine residues which are normally involved in disulfide bonding with other cysteines in the Ig molecule. Since none of the cysteines are required for the peptide to function as a tag, one or more of these cysteine residues may optionally be substituted by another amino acid residue, such as for example, serine.

Various leader sequences known in the art can be used for the efficient secretion of α2M polypeptide from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981, Proc. Natl. Acad. Sci. 78:5812-5816). Preferred leader sequences for targeting α2M polypeptide expression in bacterial cells include, but are not limited to, the leader sequences of the E. coli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffinan & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), and the Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (MacIntyre et al., 1990, Mol. Gen. Genet. 221:466-74; Kaiser et al., 1987, Science, 235:312-317).

DNA sequences encoding a desired affinity label or leader peptide, which may be readily obtained from libraries, produced synthetically, or may be available from commercial suppliers, are suitable for the practice of this invention. Such methods are well known in the art.

4.7.3. Expression Systems

Nucleotide sequences encoding an hsp or α2M and/or an antigenic molecule or an hsp-antigenic molecule or α2M-antigenic molecule fusion can be inserted into an expression vector to produce an expression construct for propagation and expression in recombinant cells. An expression construct, as used herein, refers to a nucleotide sequence encoding an hsp, α2M, and/or antigenic molecule operably associated with one or more regulatory regions which allows expression of the hsp, α2M and/or antigenic molecule in an appropriate host cell. “Operably-associated” refers to an association in which the regulatory regions and the hsp, α2M and/or antigenic molecule polypeptide sequence to be expressed are joined and positioned in such a way as to permit transcription, and ultimately, translation of the hsp, α2M and/or antigenic molecule sequence. A variety of expression vectors may be used for the expression of hsps, α2M and/or antigenic molecules, including, but not limited to, plasmids, cosmids, phage, phagemids, or modified viruses. Examples include bacteriophages such as lambda derivatives, or plasmids such as pBR322 or pUC plasmid derivatives or the Bluescript vector (Stratagene). Typically, such expression vectors comprise a functional origin of replication for propagation of the vector in an appropriate host cell, one or more restriction endonuclease sites for insertion of the hsp gene sequence or sequence encoding an antigenic molecule, and one or more selection markers.

For expression of hsps, α2M and/or antigenic molecules in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include but are not limited to those associated with the metallothionein II gene, mouse mammary tumor virus glucocorticoid responsive long terminal repeats (MMTV-LTR), the β-interferon gene, and the hsp70 gene (Williams et al., 1989, Cancer Res. 49: 2735-42; Taylor et al., 1990, Mol. Cell. Biol. 10: 165-75).

The following animal regulatory regions, which exhibit tissue specificity and have been utilized in transgenic animals, can also be used for the recombinant expression of hsps and/or antigenic molecules in cells of a particular tissue type: elastase I gene control region which is active in pancreatic acinar cells (Swift et al., 1984, Cell 38: 639-646; Ornitz et al., 1986, Cold Spring Harbor Symp. Quant. Biol. 50: 399-409; MacDonald, 1987, Hepatology 7: 425-515); insulin gene control region which is active in pancreatic beta cells (Hanahan, 1985, Nature 315: 115-122), immunoglobulin gene control region which is active in lymphoid cells (Grosschedl et al., 1984, Cell 38: 647-658; Adames et al., 1985, Nature 318: 533-538; Alexander et al., 1987, Mol. Cell. Biol. 7: 1436-1444), mouse mammary tumor virus control region which is active in testicular, breast, lymphoid and mast cells (Leder et al., 1986, Cell 45: 485-495), albumin gene control region which is active in liver (Pinkert et al., 1987, Genes Dev. 1: 268-276), alpha-fetoprotein gene control region which is active in liver (Krumlauf et al., 1985, Mol. Cell. Biol. 5: 1639-1648; Hammer et al., 1987, Science 235: 53-58; alpha 1-antitrypsin gene control region which is active in the liver (Kelsey et al., 1987, Genes Dev. 1: 161-171), beta-globin gene control region which is active in myeloid cells (Mogram et al., 1985, Nature 315: 338-340; Kollias et al., 1986, Cell 46: 89-94; myelin basic protein gene control region which is active in oligodendrocyte cells in the brain (Readhead et al., 1987, Cell 48: 703-712); myosin light chain-2 gene control region which is active in skeletal muscle (Sani, 1985, Nature 314: 283-286), and gonadotropic releasing hormone gene control region which is active in the hypothalamus (Mason et al., 1986, Science 234: 1372-1378).

The efficiency of expression of the hsp, α2M or antigenic molecule in a host cell may be enhanced by the inclusion of appropriate transcription enhancer elements in the expression vector, such as those found in SV40 virus, Hepatitis B virus, cytomegalovirus, immunoglobulin genes, metallothionein, β-actin (see Bittner et al., 1987, Methods in Enzymol. 153: 516-544; Gorman, 1990, Curr. Op. in Biotechnol. 1: 36-47).

The expression vector may also contain sequences that permit maintenance and replication of the vector in more than one type of host cell, or integration of the vector into the host chromosome. Such sequences may include but are not limited to replication origins, autonomously replicating sequences (ARS), centromere DNA, and telomere DNA. It may also be advantageous to use shuttle vectors that can be replicated and maintained in at least two types of host cells.

In addition, the expression vector may contain selectable or screenable marker genes for initially isolating or identifying host cells that contain DNA encoding an hsp, α2M and/or antigenic molecule. For long term, high yield production of hsps, α2M and/or antigenic molecules, stable expression in mammalian cells is preferred. A number of selection systems may be used for mammalian cells, including, but not limited, to the Herpes simplex virus thyrnidine kinase (Wigler et al., 1977, Cell 11: 223), hypoxanthine-guanine phosphoribosyltransferase (Szybalski and Szybalski, 1962, Proc. Natl. Acad. Sci. U.S.A. 48: 2026), and adenine phosphoribosyltransferase (Lowy et al., 1980, Cell 22: 817) genes can be employed in tk-, hgprt- or aprt-cells, respectively. Also, antimetabolite resistance can be used as the basis of selection for dihydrofolate reductase (dhfr), which confers resistance to methotrexate (Wigler et al., 1980, Natl. Acad. Sci. U.S.A. 77: 3567; O'Hare et al., 1981, Proc. Natl. Acad. Sci. U.S.A. 78: 1527); gpt, which confers resistance to mycophenolic acid (Mulligan and Berg, 1981, Proc. Natl. Acad. Sci. U.S.A. 78: 2072); neomycin phosphotransferase (neo), which confers resistance to the aminoglycoside G-418 (Colberre-Garapin et al., 1981, J. Mol. Biol. 150: 1); and hygromycin phosphotransferase (hyg), which confers resistance to hygromycin (Santerre et al., 1984, Gene 30: 147). Other selectable markers, such as but not limited to histidinol and Zeocin™ can also be used.

In order to insert the hsp or α2M coding sequence or the coding sequence of an antigenic molecule into the cloning site of a vector, DNA sequences with regulatory functions, such as promoters, must be attached to the respective coding sequences. To do this, linkers or adapters providing the appropriate compatible restriction sites may be ligated to the ends of cDNA or synthetic DNA encoding an hsp, α2M or antigenic molecule, by techniques well known in the art (Wu et al., 1987, Methods Enzymol. 152: 343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

The expression construct comprising an hsp, α2M, and/or antigenic molecule-coding sequence operably associated with regulatory regions can be directly introduced into appropriate host cells for expression and production of the hsp, α2M, and/or antigenic molecule complexes of the invention without further cloning (see e.g., U.S. Pat. No. 5,580,859). The expression constructs may also contain DNA sequences that facilitate integration of the coding sequence into the genome of the host cell, e.g., via homologous recombination. In this instance, it is not necessary to employ an expression vector comprising a replication origin suitable for appropriate host cells in order to propagate and express the hsp, α2M and/or antigenic molecule in the host cells.

Expression constructs containing cloned hsp or α2M coding sequences or coding sequences for antigenic molecules can be introduced into the mammalian host cell by a variety of techniques known in the art, including but not limited to calcium phosphate mediated transfection (Wigler et al., 1977, Cell 11: 223-232), liposome-mediated transfection (Schaefer-Ridder et al., 1982, Science 215: 166-168), electroporation (Wolff et al., 1987, Proc. Natl. Acad. Sci. 84: 3344), and microinjection (Cappechi, 1980, Cell 22: 479-488).

For long-term, high-yield production of properly processed hsp-peptide complexes, stable expression in mammalian cells is preferred. Cell lines that stably express hsps or α2M and antigenic molecules to produce hsp-peptide complexes for incorporating into the compositions of the present invention may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while the hsp or α2M and antigenic molecule is expressed continuously.

Any of the cloning and expression vectors described herein may be synthesized and assembled from known DNA sequences by techniques well known in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

Alternatively, number of viral-based expression systems may also be utilized with mammalian cells for recombinant expression of hsps, α2M and/or antigenic molecules. Vectors using DNA virus backbones have been derived from simian virus 40 (SV40) (Hamer et al., 1979, Cell 17: 725), adenovirus (Van Doren et al., 1984, Mol. Cell Biol. 4: 1653), adeno-associated virus (McLaughlin et al., 1988, J. Virol. 62: 1963), and bovine papillomas virus (Zinn et al., 1982, Proc. Natl. Acad. Sci. 79: 4897). In cases where an adenovirus is used as an expression vector, the donor DNA sequence may be ligated to an adenovirus transcription/translation control region, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing heterologous products in infected hosts (see, e.g., Logan and Shenk, 1984, Proc. Natl. Acad. Sci. U.S.A. 81: 3655-3659).

Bovine papillomavirus (BPV) can infect many higher vertebrates, including man, and its DNA replicates as an episome. A number of shuttle vectors have been developed for recombinant gene expression which exist as stable, multicopy (20-300 copies/cell) extrachromosomal elements in mammalian cells. Typically, these vectors contain a segment of BPV DNA (the entire genome or a 69% transforming fragment), a promoter with a broad host range, a polyadenylation signal, splice signals, a selectable marker, and “poisonless” plasmid sequences that allow the vector to be propagated in E. coli. Following construction and amplification in bacteria, the expression gene construct is transfected into cultured mammalian cells, for example, by the techniques of calcium phosphate coprecipitation or electroporation. For those host cells that do not manifest a transformed phenotype, selection of transformants is achieved by use of a dominant selectable marker, such as histidinol and G418 resistance. For example, BPV vectors such as pBCMGSNeo and pBCMGHis may be used to express hsps, α2M and/or antigenic molecules (Karasuyama et al., Eur. J. Immunol. 18: 97-104; Ohe et al., Human Gene Therapy 6: 325-33) which may then be transfected into a diverse range of cell types for hsp, α2M or antigenic molecule expression.

Alternatively, the vaccinia 7.5K promoter may be used (see, e.g., Mackett et al., 1982, Proc. Natl. Acad. Sci. U.S.A. 79: 7415-7419; Mackett et al., 1984, J. Virol. 49: 857-864; Panicali et al., 1982, Proc. Natl. Acad. Sci. U.S.A. 79: 4927-4931) In cases where a human host cell is used, vectors based on the Epstein-Barr virus (EBV) origin (OriP) and EBV nuclear antigen 1 (EBNA-1; a trans-acting replication factor) may be used. Such vectors can be used with a broad range of human host cells, e.g., EBO-pCD (Spickofsky et al., 1990, DNA Prot. Eng. Tech. 2: 14-18), pDR2 and λDR2 (available from Clontech Laboratories).

Recombinant hsp, α2M and/or antigenic molecule expression can also be achieved by a retrovirus-based expression system. In contrast to transfection, retroviruses can efficiently infect and transfer genes to a wide range of cell types including, for example, primary hematopoietic cells. In retroviruses such as Moloney murine leukemia virus, most of the viral gene sequences can be removed and replaced with an hsp, α2M and/or antigenic molecule coding sequence or a sequence encoding an antigenic molecule, while the missing viral functions can be supplied in trans. The host range for infection by a retroviral vector can also be manipulated by the choice of envelope used for vector packaging.

For example, a retroviral vector can comprise a 5′ long terminal repeat (LTR), a 3′ LTR, a packaging signal, a bacterial origin of replication, and a selectable marker. The ND-associated antigenic peptide DNA is inserted into a position between the 5′ LTR and 3′ LTR, such that transcription from the 5′ LTR promoter transcribes the cloned DNA. The 5′ LTR comprises a promoter, including but not limited to an LTR promoter, an R region, a U5 region and a primer binding site, in that order. Nucleotide sequences of these LTR elements are well known in the art. A heterologous promoter as well as multiple drug selection markers may also be included in the expression vector to facilitate selection of infected cells (see McLauchlin et al., 1990, Prog. Nucleic Acid Res. and Molec. Biol. 38: 91-135; Morgenstern et al., 1990, Nucleic Acid Res. 18: 3587-3596; Choulika et al., 1996, J. Virol 70: 1792-1798; Boesen et al, 1994, Biotherapy 6: 291-302; Salmons and Gunzberg, 1993, Human Gene Therapy 4:129-141; and Grossman and Wilson, 1993, Curr. Opin. in Genetics and Devel. 3: 110-114).

The recombinant cells may be cultured under standard conditions of temperature, incubation time, optical density, and media composition. Alternatively, a cell may be cultured under conditions emulating the nutritional and physiological requirements of a cell in which the hsp, α2M or antigenic molecule is endogenously expressed. Modified culture conditions and media may be used to enhance production of hsp-antigenic molecule or α2M-antigenic molecule complexes. For example, recombinant cells may be grown under conditions that promote inducible hsp expression. Any technique known in the art may be applied to establish the optimal conditions for producing hsp-antigenic molecule or α2M-antigenic molecule complexes.

4.8. Preparation of Cellular Extracts and Lysates for Use as Diluents

As described above, in certain embodiments of the invention, Diluents can be cellular lysates or extracts comprising uncomplexed hsps and/or non-specific hsp-peptide complexes. For example, cell extracts comprising hsps can simply be an unfractionated preparation of cellular proteins. In one embodiment described below in Section 4.8.1, cell extracts comprising hsps can be prepared as lysates comprising total cellular protein. In another embodiment, described in Section 4.8.2, cell extracts comprising hsps can be prepared as lysates comprising soluble cytosolic protein.

4.8.1. Preparation of Lysates Comprising Unfractionated Cellular Proteins

An exemplary, but not limiting, method that may be used to prepare unfractionated cellular proteins is as follows:

Cells, which may be tumor cells derived from a biopsy of the patient or tumor cells cultivated in vitro, or cells lines infected with a pathogenic agent, are suspended in 3 volumes of 1×Lysis buffer consisting of 30 mM sodium bicarbonate pH 7.5, and 1 mM phenyl methyl sulfonyl fluoride (PMSF). The cells may be lysed by mechanical shearing in the same Lysis buffer, which are incubated on ice for about 20 minutes to allow the cells to become hypotonically-swollen, and which are then homogenized in a dounce homogenizer until >95% cells are lysed. In other embodiments, cells resuspended in a non-hypotonic buffer, such as PBS, are lysed by freezing and thawing, or sonication. Two to five, and preferably three, freezing and thawing cycles are used, as necessary, generally until at least 90% of the cells have been lysed. Where sonication is used to lyse the cells, cells in PBS and on ice can be sonicated using a Ultrasonic Processor GE130 for 5 cycles; each cycle consisting of 10 seconds of exposure to ultrasound and thirty seconds of rest before the next cycle of sonication.

The lysate is centrifuged at 1,000×g for 10 minutes to remove unbroken cells, nuclei and other cellular debris. The clarified cell extract which comprises unfractionated cellular proteins can be dialyzed, generally for 36 hours at 40° C. (three times, 100 volumes each time) against PBS (phosphate buffered saline) or other suitable buffer, to provide the unfractionated cellular proteins of the present invention. If necessary, insoluble material in the cell extract may be removed by filtration or another low-speed centrifugation

4.8.2. Preparation of Lysates Comprising Unfractionated Cytosolic Cellular Proteins

An exemplary, but not limiting, method that may be used to prepare unfractionated cytosolic soluble proteins is as follows:

The clarified cell extract which comprises unfractionated cellular proteins prepared as described in Section 4.8.1 is recentrifuged at about 100,000×g for about one hour, and the supernatant recovered. This supernatant, which comprises unfractionated cytosolic soluble proteins of the present invention, may be dialyzed for 36 hours at 4° (three times, 100 volumes each time) against PBS (phosphate buffered saline) or other suitable buffer. If necessary, any remaining insoluble material in the preparation may be removed by filtration or further low-speed centrifugation.

4.8.3. Sources of Cell Extracts and Lysates

Diluents that are cell extracts or lysates can be prepared from any cell or tissue that does not express substantial levels of hsp-antigenic molecule complexes specific to the tumor to be treated with the Diluted Complexes comprising said Diluent, or do not express antigens associated with an agent of infectious disease when the Diluted Complexes comprising said Diluents are for the treatment or prevention of an infectious disease caused by the agent. As used herein, the term “substantial levels of specific antigens” refers to levels of antigens sufficient to be immunogenic—i.e., capable of eliciting, stimulating, enhancing and/or sustaining with specificity an immune response in a subject to whom they are administered.

Cells whose lysates or extracts are suitable for use as Diluents in the present invention can be any cells except a cell that is identical to the cell from which an hsp-peptide complex that is a Specific Complex was prepared. For example, a lysate or extract of a cell of different or organ type than that from which the Specific Complex was prepared can be used as a Diluent, as can a healthy counterpart of the diseased cell or organ type from which the Specific Complex was prepared.

In one embodiment, the Specific Complex is prepared from a primary (non-immortalized) diseased cell of a patient, and the Diluent is a lysate prepared from an immortalized cell culture of the patient, which immortalized cell culture possesses distinct antigenic properties from the diseased cell from which the Specific Complex was prepared.

In another embodiment, the Diluent is a lysate prepared from a cell of a different species origin than that from which the Specific Complex was prepared. Such cells can be of the same or different cell, tissue or organ type as the undiseased counterpart of the cell from which the Specific Complex is obtained.

4.9. Prevention and Treatment of Cancer and Infectious Disease

In accordance with the invention, a composition of the invention which is a Diluted Complex, comprising a Specific Complex and a Diluent, i.e., non-Specific hsp or hsp-peptide complex, is administered to a human subject with cancer or an infectious disease. In one embodiment, “treatment” or “treating” refers to an amelioration of cancer or an infectious disease, or at least one discernible symptom thereof. In another embodiment, “treatment” or “treating” refers to an amelioration of at least one measurable physical parameter associated with cancer or an infectious disease, not necessarily discernible by the subject. In yet another embodiment, “treatment” or “treating” refers to inhibiting the progression of a cancer or an infectious disease, either physically, e.g., stabilization of a discernible symptom, physiologically, e.g., stabilization of a physical parameter, or both. In yet another embodiment, “treatment” or “treating” refers to delaying the onset of a disease or disorder.

In certain embodiments, the compositions of the present invention are administered to a human subject as a preventative measure against such cancer or an infectious disease. As used herein, “prevention” or “preventing” refers to a reduction of the risk of acquiring a given cancer or infectious disease. In one mode of the embodiment, the compositions of the present invention are administered as a preventative measure to a human subject having a genetic predisposition to a cancer. In another mode of the embodiment, the compositions of the present invention are administered as a preventative measure to a subject facing exposure to carcinogens including but not limited to chemicals and/or radiation, or to a subject facing exposure to an agent of an infectious disease.

4.9.1. Target Infectious Diseases

Infectious diseases that can be treated or prevented by the methods of the present invention are caused by infectious agents including, but not limited to, viruses, bacteria, fungi protozoa and parasites.

Viral diseases that can be treated or prevented by the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

Bacterial diseases that can be treated or prevented by the methods of the present invention are caused by bacteria including, but not limited to, mycobacteria rickettsia, mycoplasma, neisseria and legionella.

Protozoal diseases that can be treated or prevented by the methods of the present invention are caused by protozoa including, but not limited to, leishmania, kokzidioa, and trypanosoma.

Parasitic diseases that can be treated or prevented by the methods of the present invention are caused by parasites including, but not limited to, chlamydia and rickettsia.

4.9.2. Target Cancers

Cancers that can be treated or prevented by the methods of the present invention include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcomna, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease. Specific examples of such cancers are described in the sections below.

In a specific embodiment the cancer is metastatic. In another specific embodiment, the patient having a cancer is immunosuppressed by reason of having undergone anti-cancer therapy (e.g., chemotherapy radiation) prior to administration of the hsp-peptide complexes or administration of the hsp-sensitized APC.

There are many reasons why immunotherapy as provided by the present invention is desired for use in cancer patients. First, if cancer patients are immunosuppressed, surgery with anesthesia and subsequent chemotherapy may worsen the immunosuppression. With appropriate immunotherapy in the preoperative period, this immunosuppression may be prevented or reversed. This could lead to fewer infectious complications and to accelerated wound healing. Second, tumor bulk is minimal following surgery and immunotherapy is most likely to be effective in this situation. A third reason is the possibility that tumor cells are shed into the circulation at surgery and effective immunotherapy applied at this time can eliminate these cells.

The preventive and therapeutic methods of the invention are directed at enhancing the immunocompetence of the cancer patient either before surgery, at or after surgery, and to induce tumor-specific immunity to cancer cells, with the objective being inhibition of cancer, and with the ultimate clinical objective being total cancer regression and eradication.

4.9.2.1 Colorectal Cancer Metastatic to the Liver

It has been estimated that approximately 226,600 Americans will be diagnosed with cancers of the digestive tract in 2000. Most notably, the colon will be the primary site for approximately 93,800 of these cases and the rectum the primary site for another approximately 36,400 cases. Further, it is predicted that approximately 47,700 will die of colon cancer and another 8,600 will die of rectal cancer (Cancer Facts & Figures. 2000, American Cancer Society (ACS), Atlanta, Ga., 2000). 80 percent of patients who die of colon or rectal cancer have metastatic disease involving the liver. Most metastatic tumors of the liver are from gastrointestinal primaries. Unfortunately, the natural history of metastatic liver lesions carries a grave prognosis and systemic chemotherapy regimens have been unable to induce significant response rates or alter length of survival (Drebin, J. A., et al., in Current Therapy In Oncology, ed. J. E. Niederhuber, B. C. Decker, Mosby, 1993, p.426).

Colorectal cancer initially spreads to regional lymph nodes and then through the portal venous circulation to the liver, which represents the most common visceral site of metastasis. The symptoms that lead patients with colorectal cancer to seek medical care vary with the anatomical location of the lesion. For example, lesions in the ascending colon frequently ulcerate, which leads to chronic blood loss in the stool.

Radical resection offers the greatest potential for cure in patients with invasive colorectal cancer. Before surgery, the CEA titer is determined. Radiation therapy and-chemotherapy are used in patients with advanced colorectal cancer. Results with chemotherapeutic agents (e.g., 5-fluorouracil) are mixed and fewer than 25 percent of patients experience a greater than 50 percent reduction in tumor mass (Richards, 2d., F., et al., 1986, J. Clin. Oncol. 4:565).

Patients with widespread metastases have limited survival and systemic chemotherapy has little impact in this group of patients. In addition, systemically administered chemotherapy is often limited by the severity of toxicities associated with the various agents, such as severe diarrhea, mucositis and/or myelosuppression. Other techniques, including hepatic radiation, systemic chemotherapy, hepatic arterial ligation, tumor embolization and immunotherapy have all been explored, but, for the most part, have proven ineffectual in prolonging patient survival.

In a specific embodiment, the present invention provides compositions and methods for enhancing tumor specific immunity in individuals suffering from colorectal cancer metastasized to the liver, in order to inhibit the progression of the neoplastic disease. Preferred methods of treating these neoplastic diseases comprise administering a Diluted Complex in which the Specific Complex comprises autologous hsp bound to peptide complexes, which elicits tumor-specific immunity against the tumor cells. In one embodiment, the Diluent is also autologous and is prepared from a non-cancerous tissue. In another embodiment, the Diluent is allogeneic, for example a recombinantly expressed hsp. Most specifically, the use of a composition of the invention, whose Specific Complex comprises gp96, can result in nearly complete inhibition of liver cancer growth in cancer patients, without inducing toxicity and thus providing a dramatic therapeutic effect.

Accordingly, as an example of the method of the invention, a Diluted Complex in which the Specific Complex comprises gp96 associated with an antigenic molecule is administered to a patient diagnosed with colorectal cancer, with or without liver metastasis, via one of many different routes of administration, the preferred route being intradermally at different anatomical sites, e.g., left arm, right arm, left belly, right belly, left thigh, right thigh, etc. The site of injection is varied for each weekly injection. Exemplary primary and metastatic cancers that can be prevented or treated according to the methods of the invention are described in detail in the sections which follow and by way of example, infra.

4.9.2.2 Hepatocellular Carcinoma

Hepatocellular carcinoma is generally a disease of the elderly in the United States. Although many factors may lead to hepatocellular carcinoma, the disease is usually limited to those persons with preexisting liver disease. Approximately 60 to 80 percent of patients in the United States with hepatocellular carcinoma have a cirrhotic liver and about four percent of individuals with a cirrhotic liver eventually develop hepatocellular carcinoma (Niederhuber, J. E., (ed.), 1993, Current Therapy in Oncology, B. C. Decker, Mosby). The risk is highest in patients whose liver disease is caused by inherited hemochromatosis or hepatic B viral infection (Bradbear, R. A., et al., 1985, J. Natl. Cancer Inst. 75:81; Beasley, R. P., et al., 1981, Lancet 2:1129); hepatitis C virus infection has also emerged as a risk factor in the past decade (Colombo, 1999, Baillieres Best Pract Res Clin Gastroenterol 13(4):519-28). Other causes of cirrhosis that can lead to hepatocellular carcinoma include alcohol abuse and hepatic fibrosis caused by chronic administration of methotrexate. The most frequent symptoms of hepatocellular carcinoma are the development of a painful mass in the right upper quadrant or epigastrium, accompanied by weight loss. In patients with cirrhosis, the development of hepatocellular carcinoma is preceded by ascites, portal hypertension and relatively abrupt clinical deterioration. In most cases, abnormal values in standard liver function tests such as serum aminotransferase and alkaline phosphatase are observed.

CT scans of the liver are used to determine the anatomic distribution of hepatocellular carcinoma and also provide orientation for percutaneous needle biopsy. Approximately 70 percent of patients with hepatocellular carcinoma have an elevated serum alpha-fetoprotein concentration (McIntire, K. R., et al., 1975, Cancer Res. 35:991) and its concentration correlates with the extent of the disease.

Radical resection offers the only hope for cure in patients with hepatocellular carcinoma. Such operative procedures are associated with five-year survival rates of 12 to 30 percent. Liver transplantation may improve survival of some younger individuals. However, most patients are not surgical candidates because of extensive cirrhosis multifocal tumor pattern or scarcity of compatible donor organs.

Chemotherapeutic agents have been administered either by intravenous route or through an intrahepatic arterial catheter. Such therapy has sometimes been combined with irradiation to the liver. Reductions in the size of measurable tumors of 50% or more have been reported in some patients treated with either systemic doxorubicin or 5-fluorouracil. However, chemotherapy often induces immunosuppression and rarely causes the tumor to disappear completely and the duration of response is short. The prognosis for patients with hepatocellular carcinoma is negatively correlated with cirrhosis and metastases to the lungs or bone. Median survival for patients is only four to six months. In a specific embodiment, the present invention provides compositions and methods for enhancing specific immunity in individuals suffering from hepatocellular carcinoma in order to inhibit the progression of the neoplastic disease and ultimately irradiate all preneoplastic and neoplastic cells.

4.9.2.3 Breast Cancer

Another specific aspect of the invention relates to the treatment of breast cancer. The American Cancer Society estimated that in 2000, 184,200 American women will be diagnosed with breast cancer and 41,200 will succumb to the disease (Cancer Facts & Figures. 2000, American Cancer Society (ACS), Atlanta, Ga., 2000). This makes breast cancer the second major cause of cancer death in women, ranking just behind lung cancer. The treatment of breast cancer presently involves surgery, radiation, hormonal therapy and/or chemotherapy. Consideration of two breast cancer characteristics, hormone receptors and disease extent, has governed how hormonal therapies and standard-dose chemotherapy are sequenced to improve survival and maintain or improve quality of life. A wide range of multidrug regimens have been used as adjuvant therapy in breast cancer patients, including, but not limited to combinations of 2 cyclophosphamide, doxorubicin, vincristine methotrexate, 5-fluorouracil and/or leucovorin. In a specific embodiment, the present invention provides hsp compositions of Diluted Complexes and methods for enhancing specific immunity to preneoplastic and neoplastic mammary cells in women. The present invention also provides compositions of Diluted Complexes and methods for preventing the development of neoplastic cells in women at enhanced risk for breast cancer, and for inhibiting cancer cell proliferation and metastasis. These compositions can be applied alone or in combination with each other or with biological response modifiers.

4.9.3. Autologous Embodiment

The specific immunogenicity of hsps derives not from hsps per se, but from the peptides bound to them. In a preferred embodiment of the invention, the Specific Complexes in compositions of the inventions for use as cancer vaccines are autologous complexes, thereby circumventing two of the most intractable hurdles to cancer immunotherapy. First is the possibility that human cancers, like cancers of experimental animals, are antigenically distinct. In a preferred embodiment of the present invention, the hsps of the Specific Complex chaperone antigenic peptides of the cancer cells from which they are derived and circumvent this hurdle. Second, most current approaches to cancer immunotherapy focus on determining the CTL-recognized epitopes of cancer cell lines. This approach requires the availability of cell lines and CTLs against cancers. These reagents are unavailable for an overwhelming proportion of human cancers. In an embodiment of the present invention directed to the use of autologous Specific Complexes of hsp-peptides, cancer immunotherapy does not depend on the availability of cell lines or CTLs nor does it require definition of the antigenic epitopes of cancer cells. These advantages make autologous hsps bound to peptide complexes attractive immunogens against cancer. In one mode of this autologous embodiment, the Diluents are also autologous to the individual to whom they are administered, but are derived from an alternative cell source that is not expected to comprise the antigenic molecules of the Specific Complexes. In another mode of this embodiment, the Diluents can be prepared from a cell culture line that expresses a heat shock protein encoded by the individual to whom the composition of the invention is to be administered. In yet another mode of this embodiment, the heat shock proteins of the Diluent may be allogeneic to the individual to whom a composition of the invention is to be administered.

4.10. Determination of Immunogenicity of Hsp- and α2M-peptide Complexes

Optionally, the Specific Complexes and the Diluted Complexes of the invention can be assayed for immunogenicity using any method known in the art. The Diluents can also be assayed, to ensure confirm their lack of antigenicity against the antigen source of interest or as control complexes. By way of example but not limitation, one of the following procedures can be used. In a preferred embodiment, the ELISPOT assay is used (see, infra, Section 4.10.4)

4.10.1. The MLTC Assay

Briefly, mice are injected with an amount of the Specific or Diluted Complex, using any convenient route of administration. As a negative control, other mice are injected with, e.g., hsp-peptide or α2M-peptide complexes that are to be used as non-specific or Diluents. Cells known to contain specific antigens, e.g. tumor cells or cells infected with an agent of an infectious disease, may act as a positive control for the assay. The mice are injected twice, 7-10 days apart. Ten days after the last immunization, the spleens are removed and the lymphocytes released. The released lymphocytes may be re-stimulated subsequently in vitro by the addition of dead cells that expressed the antigen of interest.

For example, 8×10⁶ immune spleen cells may be stimulated with 4×10⁴ mitomycin C treated or γ-irradiated (5-10,000 rads) cells containing the antigen of interest (or cells transfected with an appropriate gene, as the case may be) in 3 ml RPMI medium containing 10% fetal calf serum. In certain cases 33% secondary mixed lymphocyte culture supernatant may be included in the culture medium as a source of T cell growth factors (See-Glasebrook, et al., 1980, J Exp. Med. 151:876). To test the primary cytotoxic T cell response after immunization, spleen cells may be cultured without stimulation. In some experiments spleen cells of the immunized mice may also be re-stimulated with antigenically distinct cells, to determine the specificity of the cytotoxic T cell response.

Six days later the cultures are tested for cytotoxicity in a 4 hour ⁵¹Cr-release assay (See, Palladino, et al., 1987, Cancer Res. 47:5074-5079 and Blachere, at al., 1993, J. Immunotherapy 14:352-356). In this assay, the mixed lymphocyte culture is added to a target cell suspension to give different effector:target (E:T) ratios (usually 1:1 to 40:1). The target cells are prelabelled by incubating 1×10⁶ target cells in culture medium containing 20 mCi ⁵¹Cr/ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelletted by centrifugation at 200 g for 5 minutes. The amount of ⁵ Cr released into the supernatant is measured by a gamma counter. The percent cytotoxicity is measured as cpm in the test sample minus spontaneously released cpm divided by the total detergent released cpm minus spontaneously released cpm.

In order to block the MHC class I cascade a concentrated hybridoma supernatant derived from K-44 hybridoma cells (an anti-MHC class I hybridoma) is added to the test samples to a final concentration of 12.5%.

4.10.2. CD4+ T Cell Proliferation Assay

Primary T cells are obtained from spleen, fresh blood, or CSF and purified by centrifugation using FICOLL-PAQUE PLUS (Pharmacia, Upsalla, Sweden) essentially as described by Kruse and Sebald, 1992, EMBO J. 11: 3237-3244. The peripheral blood mononuclear cells are incubated for 7-10 days with a lysate of cells expressing an antigenic molecule. Antigen presenting cells may, optionally be added to the culture 24 to 48 hours prior to the assay, in order to process and present the antigen in the lysate. The cells are then harvested by centrifugation, and washed in RPMI 1640 media (GibcoBRL, Gaithersburg, Md.). 5×10⁴ activated T cells/well are in RPMI 1640 media containing 10% fetal bovine serum, 10 mM HEPES, pH 7.5, 2 mM L-glutamine, 100 units/ml penicillin G, and 100 μg/ml streptomycin sulphate in 96 well plates for 72 hrs at 37° C., pulsed with 1 μCi ³H-thymidine (DuPont NEN, Boston, Mass.)/well for 6 hrs, harvested, and radioactivity measured in a TOPCOUNT scintillation counter (Packard Instrument Co., Meriden, Conn.).

4.10.3. Antibody Response Assay

In a certain embodiment of the invention, the immunogenicity of an hsp-peptide or α2M-peptide complex is determined by measuring antibodies produced in response to the vaccination with the complex. In one mode of the embodiment, microtitre plates (96-well Immuno Plate II, Nunc) are coated with 50 μl/well of a 0.75 μg/ml solution of a purified, non-hsp- or α2M-complexed form of the peptide used in the vaccine (e.g. Aβ42) in PBS at 4° C. for 16 hours and at 20° C. for 1 hour. The wells are emptied and blocked with 200 μl PBS-T-BSA (PBS containing 0.05% (v/v) TWEEN 20 and 1% (w/v) bovine serum albumin) per well at 20° C. for 1 hour, then washed 3 times with PBS-T. Fifty μl/well of plasma or CSF from a vaccinated animal (such as a model mouse or a human patient) is applied at 20° C. for 1 hour, and the plates are washed 3 times with PBS-T. The anti-peptide antibody activity is then measured calorimetrically after incubating at 20° C. for 1 hour with 50 μl/well of sheep anti-mouse or anti-human immunoglobulin, as appropriate, conjugated with horseradish peroxidase (Amersham) diluted 1:1,500 in PBS-T-BSA and (after 3 further PBS-T washes as above) with 50 μl of an o-phenylene diamine (OPD)-H₂O₂ substrate solution. The reaction is stopped with 150 μl of 2M H₂SO₄ after 5 minutes and absorbance is determined in a Kontron SLT-210 photometer (SLT Lab-instr., Zurich, Switzerland) at 492 nm (ref 620 nm).

4.10.4. Cytokine Detection Assays

The CD4+ and CD8+ T cell proliferative response to the Diluted Complexes of the invention may be measured by detection and quantitation of the levels of specific cytokines. In one embodiment, for example, intracellular cytokines may be measured using an IFN-γ detection assay to test for immunogenicity of a complex of the invention. In an example of this method, peripheral blood mononuclear cells from a subject treated with a Diluted Complex are stimulated with peptide antigens of a given tumor or with peptide antigens of an agent of infectious disease. Cells are then stained with T cell-specific labeled antibodies detectable by flow cytometry, for example FITC-conjugated anti-CD8 and PerCP-labeled anti-CD4 antibodies. After washing, cells are fixed, permeabilized, and reacted with dye-labeled antibodies reactive with human IFN-γ (PE- anti-IFN-γ). Samples are analyzed by flow cytometry using standard techniques.

Alternatively, a filter immunoassay, the enzyme-linked immunospot assay (ELISPOT) assay, may be used to detect specific cytokines surrounding a T cell. In one embodiment, for example, a nitrocellulose-backed microtiter plate is coated with a purified cytokine-specific primary antibody, i.e., anti-IFN-γ, and the plate is blocked to avoid background due to nonspecific binding of other proteins. A sample of mononuclear blood cells, containing cytokine-secreting cells, obtained from a subject treated with a Diluted Complex, which sample is diluted onto the wells of the microtitre plate. A labeled, e.g., biotin-labeled, secondary anti-cytokine antibody is added. The antibody cytokine complex can then be detected, i.e. by enzyme-conjugated streptavidin—cytokine-secreting cells will appear as “spots” by visual, microscopic, or electronic detection methods.

4.10.5. Tetramer Assay

In another embodiment, the “tetramer staining” assay (Altman et al., 1996, Science 274: 94-96) may be used to identify antigen-specific T-cells. For example, in one embodiment, an MHC molecule containing a specific peptide antigen, such as a tumor-specific antigen, is multimerized to make soluble peptide tetramers and labeled, for example, by complexing to streptavidin. The MHC-peptide antigen complex is then mixed with a population of T cells obtained from a subject treated with a Diluted Complex. Biotin is then used to stain T cells which express the antigen of interest, i e., the tumor-specific antigen.

4.11. Combination With Adoptive Immunotherapy

Adoptive immunotherapy refers to a therapeutic approach for treating cancer or infectious diseases in which immune cells are administered to a host with the aim that the cells mediate either directly or indirectly specific immunity to tumor cells and/or antigenic components or regression of the tumor or treatment of infectious diseases, as the case may be. (See U.S. Pat. No. 5,985,270, issued Nov. 16, 1999, which is incorporated by reference herein in its entirety.) As an optional step, in accordance with the methods described herein, APC are sensitized with hsps or α2M complexed with antigenic (or immunogenic) molecules and used in adoptive immunotherapy.

In a specific embodiment, therapy by administration of Diluted Complexes, using any desired route of administration, may optionally be combined with adoptive immunotherapy using APC sensitized with hsp-peptide or α2M-peptide complexes. The sensitized APC can be administered alone, in combination with the Diluted complexes, or before or after administration of the Diluted Complexes. Furthermore, the mode of administration can be varied, including but not limited to, e.g., subcutaneously, intravenously or intramuscularly, although intradermally is preferred.

4.11.1. Obtaining Antigen-presenting Cells

The antigen-presenting cells, including but not limited to macrophages, dendritic cells and B-cells, are preferably obtained by production in vitro from stem and progenitor cells from human peripheral blood or bone marrow as described by Inaba, K., et al., 1992, J. Exp. Med. 176:1693-1702. Dendritic cells can be obtained by any of various methods known in the art. By way of example but not limitation, dendritic cells can be obtained by the methods described in Sallusto et al., 1994, J Exp Med 179:1109-1118 and Caux et al., 1992, Nature 360, 258-261 which are incorporated herein by reference in their entireties. In a preferred aspect, human dendritic cells obtained from human blood cells are used.

APC can be obtained by any of various methods known in the art. In one aspect, human macrophages are used, obtained from human blood cells. By way of example but not limitation, macrophages can be obtained as follows:

Mononuclear cells are isolated from peripheral blood of a patient (preferably the patient to be treated), by Ficoll-Hypaque gradient centrifugation and are seeded on tissue culture dishes which are pre-coated with the patient's own serum or with other AB+ human serum. The cells are incubated at 37° C. for 1 hour, then non-adherent cells are removed by pipetting. To the adherent cells left in the dish, is added cold (4° C.) 1 mM EDTA in phosphate-buffered saline and the dishes are left at room temperature for 15 minutes. The cells are harvested, washed with RPMI buffer and suspended in RPMI buffer. Increased numbers of macrophages may be obtained by incubating at 37° C. with macrophage-colony stimulating factor (M-CSF).

4.11.2. Sensitization Antigen Presenting Cells with Hsp-peptide Complexes

APC are sensitized with hsp or α2M bound to antigenic molecules preferably by incubating the cells in vitro with the complexes. The APC are sensitized with the Specific or Diluted Complexes of the invention by incubating in vitro with the complexes at 37° C. for 15 minutes to 24 hours. By way of example but not limitation, 4×10⁷ macrophages can be incubated with 10 microgram gp96-peptide complexes per ml or 100 microgram hsp90-peptide complexes per ml at 37° C. for 15 minutes-24 hours in 1 ml plain RPMI medium. The cells are washed three times and resuspended in a physiological medium preferably sterile, at a convenient concentration (e.g., 1 ⁷/ml) for injection in a patient. Preferably, the patient into which the sensitized APCs are injected is the patient from which the APC were originally isolated (autologous embodiment).

Optionally, the ability of sensitized APC to stimulate, for example the antigen-specific, class I-restricted cytotoxic T-lymphocytes (CTL) can be monitored by their ability to stimulate CTLs to release tumor necrosis factor, and by their ability to act as targets of such CTLs.

4.11.3. Reinfusion of Sensitized APC

APCs sensitized with Specific or Diluted Complexes are reinfused into the patient systemically, preferably intradermally, by conventional clinical procedures. These activated cells are reinfused, preferentially by systemic administration into the autologous patient. Patients generally receive from about 10⁶ to about 10¹² sensitized macrophages, depending on the condition of the patient. In some regimens, patients may optionally receive in addition a suitable dosage of a biological response modifier including but not limited to the cytokines IFN-α, IFN-γ, IL-2, IL-4, IL-6, TNF or other cytokine growth factor.

4.12. Passive Immunotherapy

The compositions of the invention can also be used for passive immunotherapy against cancers and infectious diseases. Passive immunity is the short-term protection of a host, achieved by the administration of pre-formed antibody directed against a heterologous organism. For example, compositions of the invention comprising Diluted Complexes obtained from cells infected with an infectious organism may be used to elicit an immune response in a subject, preferably after covalent cross-linking of the specific or Diluted Complexes. The sera removed from the subject and used for treatment or prevention of a disease caused by the infectious organism in another subject. Optionally, specific antibodies in the sera can be purified, for example by affinity purification.

4.13. Combination Therapy for Cancer Treatment

The present compositions can be administered together with treatment with irradiation or one or more chemotherapeutic agents. For irradiation treatment, the irradiation can be gamma rays or X-rays. For a general overview of radiation therapy, see Hellman, Chapter 16: Principles of Cancer Management: Radiation Therapy, 6th edition, 2001, DeVita et al., eds., J.B. Lippencott Company, Philadelphia. Useful chemotherapeutic agents include methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a specific embodiment, a composition of the invention is administered concurrently with radiation therapy or one or more chemotherapeutic agents. In another specific embodiment, chemotherapy or radiation therapy is administered prior or subsequent to administration of a present composition, preferably at least an hour, five hours, 12 hours, a day, a week, a month, more preferably several months (e.g., up to three months), subsequent to administration of a composition of the invention.

4.14. Formulation, Administration & Kits

4.14.1. Formulation and Administration

As discussed above, the compositions of the present invention comprise an immunogenic mixture of (i) an hsp or α2M molecular complex and (ii) hsp, α2M , or an hsp or α2M molecular complex, namely (i) a Specific Complex and (ii) a Diluent. Each complex may be of a uniform nature or may comprise admixture of heat shock protein-peptide complexes or α2M molecular complexes. Where both the Specific Complex and Diluent comprise hsps, the Specific Complex and Diluent may each comprise primarily the same heat shock protein or different heat shock proteins. The Specific Complexes and Diluents may each or both be prepared by purification from an in vivo source, for example from diseased tissue in the case of Specific Complexes and from non-diseased tissue in the case of the Diluent, or from an in vitro source, for example by recombinant expression of the heat shock proteins and associated peptides. The compositions can be prepared by mixing a preparation of Specific Complex and a preparation of Diluent. The Diluted Complexes of the invention may comprise any mass ratio of Specific Complexes to Diluents, preferably ranging from 1:1 to 1:1000, more preferably ranging from 1:1 to 1:100, e.g., 1:1, 1:2, 1:5, 1:10, 1;20, 1:50, 1:100, etc.

The amount of the Diluted Complex administered will vary depending on the amount of Specific Complex in the Diluted Complex, as well as the components of each. A dosage can be measured in terms of the Diluted Complex or in terms of the Specific Complex component of the Diluted complex.

The dosage of Diluted Complex is preferably 1-100 μg where the Specific Complex comprises gp96, hsp70, hsp 110 or grp 170, and is more preferably 2-50 μg, and yet most preferably about 5-25 μg.

Where the Specific Complex comprises hsp90, the dosage of Diluted Complex is preferably 10-500 μg, more preferably 20-400 μg, and yet more preferably 50-250 μg.

Where the Specific Complex comprises α2M, the dosage of Diluted Complex is preferably 1 μg-10 mg, more preferably 2 μg-5 mg, more preferably 5 μg-500 μg, and is most preferably 5-250 μg.

Where the Specific Complex comprises calreticulin, the dosage of Diluted Complex is preferably 0.5-50 μg, more preferably 1-25 μg, yet more preferably 2 μg-15 μg, and is most preferably 2.5-10 μg.

With reference to the amount of a Specific Complex, a dosage of Diluted Complex comprises 1-10 μg, 2-5 μg, 5-10 μg, or 10-20 μg of a Specific Complex comprising gp96, hsp 70, hsp 110 or grp170, regardless of the total amount of Diluted Complex. In specific modes of the embodiment, a dosage of Diluted Complex comprises approximately 1 μg, 2 μg, 3 μg, 4 μg, 5 μg, or 10 μg of a Specific Complex comprising gp96, hsp 70, hsp 110 or grp170.

In other embodiments, a dosage of Diluted Complex comprises 5-50 μg, 10-100 μg, 20-50 μg or 50-100 μg of a Specific Complex comprising hsp 90, regardless of the total amount of Diluted Complex. In specific modes of the embodiment, a dosage of Diluted Complex comprises approximately 5 μg, 7.5 μg, 10 μg, 12.5 μg, 15 μg, 20 μg, 30 μg, 40 μg or 50 μg of a Specific Complex comprising gp96, hsp 70, hsp110 or grp170.

In yet other embodiments, a dosage of Diluted Complex comprises 5-50 μg, 10-100 μg, 20-50 μg or 50-100 μg of a Specific Complex comprising hsp 90, regardless of the total amount of Diluted Complex. In specific modes of the embodiment, a dosage of Diluted Complex comprises approximately 5 μg, 7.5 μg, 10 μg, 12.5 μg, 15 μg, 20 μg, 30 μg, 40 μg or 50 μg of a Specific Complex comprising gp96, hsp 70, hsp 110 or grp170.

In yet other embodiments, a dosage of Diluted Complex comprises 0.5-5 μg, 1-2.5 μg, 2.5-5 μg, or 5-10 μg of a Specific Complex comprising gp96, hsp 70, hsp 110 or grp170 regardless of the total amount of Diluted Complex. In specific modes of the embodiment, a dosage of Diluted Complex comprises approximately 0.5, 1 μg, 1.5, 2 μg, 2.5, or 5 μg of a Specific Complex comprising calreticulin.

Table 1 below provides exemplary combinations of specific and diluent hsp and/or α2M amount for each therapeutic or prophylactic administration of the compositions of the invention:

TABLE 1
Exemplary combinations of Specific Complexes and Diluents and
resulting total doses of Diluted Complexes for therapeutic or preventative administration.

| Ratio of Specific hsp | | | | | |
|---|---|---|---|---|---|
| or α2M to total hsp | Specific hsp | | | | |
| and α2M in Diluted | (μg) | | Diluent hsp (μg) | | Total |
| Complex | or α2M (μg) | + | or α2M (μg) | = | dose (μg) |
| 1:100 | 0.01 | | 0.99 | | 1.0 |
| 1:20 | 0.05 | | 0.95 | | 1.0 |
| 1:10 | 0.10 | | 0.90 | | 1.0 |
| 1:1 | 0.50 | | 0.50 | | 1.0 |
| 1:200 | 0.05 | | 9.95 | | 10.0 |
| 1:10 | 1.0 | | 9.0 | | 10.0 |
| 1:5 | 2.0 | | 8.0 | | 10.0 |
| 1:1 | 5.0 | | 5.0 | | 10.0 |

The Diluted Complexes of the invention may be formulated into pharmaceutical preparations for administration to mammals, preferably humans, for treatment or prevention of cancer or infectious diseases. Compositions comprising a Diluted Complex of the invention formulated in a compatible pharmaceutical carrier may be prepared, packaged, and labelled for treatment of the indicated tumor(s), such as human sarcomas and carcinomas,. Alternatively, pharmaceutical compositions may be formulated for treatment of appropriate infectious diseases.

Drug solubility and the site of absorption are factors which should be considered when choosing the route of administration of a therapeutic agent. In an embodiment of the invention, hsp-peptide complexes may be administered using any desired route of administration, preferably subcutaneously and more preferably intradermally. Advantages of intradermal administration rapid absorption.

If the Diluted Complex is water-soluble, then it may be formulated in an appropriate buffer, for example, phosphate buffered saline or other physiologically compatible solutions, preferably sterile. Alternatively, if the resulting Diluted Complex has poor solubility in aqueous solvents, then it may be formulated with a non-ionic surfactant such as Tween, or polyethylene glycol. Thus, the Diluted Complexes and their physiologically acceptable solvates may be formulated for administration by inhalation or insufflation (either through the mouth or the nose) or oral, buccal, parenteral, or rectal administration or, in the case of tumors, directly injected into a solid tumor.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the Diluted Complexes.

For buccal administration, the compositions may take the form of tablets or lozenges formulated in conventional manner.

The Diluted Complexes may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The Diluted Complexes may also be formulated in rectal compositions such as suppositories or retention enemas, e.g., containing conventional suppository bases such as cocoa butter or other glycerides.

In addition to the formulations described previously, the Diluted Complexes may also be formulated as a depot preparation. Such long acting formulations may be administered by implantation (for example, subcutaneously or intramuscularly) or by intramuscular injection. Thus, for example, the Diluted Complexes may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

For administration by inhalation, the Diluted Complexes for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the Diluted Complexes and a suitable powder base such as lactose or starch.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

4.14.2. Kits

The invention also provides kits for carrying out the methods and/or therapeutic regimens of the invention. In one embodiment, such kits comprise in one container a Diluent for combining with a Specific Complex to be isolated from a specific patient for autologous administration. Optionally, a purified hsp or α2M for complexing to an antigenic molecule of choice is further provided in a second container.

In another embodiment, such kits comprise in one or more containers therapeutically or prophylactically effective amounts of the Diluted Complexes, preferably purified, in pharmaceutically acceptable form. The kits optionally further comprise in a second container APCs, preferably purified. The APCs may be sensitized. Alternatively, the kit may provide in yet another container a Specific or Diluted Complexes for sensitizing the APCs.

The hsp-peptide or α2M-peptide complex in a container of a kit of the invention may be in the form of a pharmaceutically acceptable solution, e.g., in combination with sterile saline, dextrose solution, or buffered solution, or other pharmaceutically acceptable sterile fluid. Alternatively, the hsp or α2M preparations may be lyophilized or desiccated; in this instance, the kit optionally further comprises in a container a pharmaceutically acceptable solution (e.g., saline, dextrose solution, etc.), preferably sterile, to reconstitute the hsps, α2M, or hsp- or α2M-containing complexes to form a solution for injection purposes.

In another embodiment, a kit of the invention optionally comprises a reagent that promotes formation of a covalent complex between the antigenic molecule and the hsp or α2M, for example a cross-linking reagent such as glutaraldehyde.

In another embodiment, a kit of the invention further comprises a needle or syringe, preferably packaged in sterile form, for injecting the complex, and/or a packaged alcohol pad. Instructions are optionally included for administration of hsp-peptide complexes by a clinician or by the patient.

4.15. Monitoring of Effects During Cancer Prevention and Immunotherapy with Hsp-peptide Complexes

The effect of immunotherapy with Diluted Complexes on development and progression of neoplastic diseases can be monitored by any methods known to one skilled in the art, including but not limited to measuring: a) delayed hypersensitivity as an assessment of cellular immunity; b) activity of cytolytic T-lymphocytes in vitro; c) levels of tumor specific antigens, e.g., carcinoembryonic (CEA) antigens; d) changes in the morphology of tumors using techniques such as a computed tomographic (CT) scan; and e) changes in levels of putative biomarkers of risk for a particular cancer in individuals at high risk, and f) changes in the morphology of tumors using a sonogram.

4.15.1. Delayed Hypersensitivity Skin Test Delayed hypersensitivity skin tests are of great value in the overall immunocompetence and cellular immunity to an antigen. Inability to react to a battery of common skin antigens is termed anergy (Sato, T., et al., 1995, Clin. Immunol. Pathol. 74:35-43).

Proper technique of skin testing requires that the antigens be stored sterile at 4° C., protected from light and reconstituted shorted before use. A 25- or 27-gauge needle ensures intradermal, rather than subcutaneous, administration of antigen. Twenty-four and 48 hours after intradermal administration of the antigen, the largest dimensions of both erythema and induration are measured with a ruler. Hypoactivity to any given antigen or group of antigens is confirmed by testing with higher concentrations of antigen or, in ambiguous circumstances, by a repeat test with an intermediate test.

4.15.2. Activity of Cytolytic T-lymphocytes In Vitro

8×10⁶ peripheral blood derived T lymphocytes isolated by the Ficoll-Hypaque centrifugation gradient technique, are restimulated with 4×10⁴ mitomycin C treated tumor cells in 3 ml RPMI medium containing 10% fetal calf serum. In some experiments, 33% secondary mixed lymphocyte culture supernatant or IL-2, is included in the culture medium as a source of T cell growth factors.

In order to measure the primary response of cytolytic T-lymphocytes after immunization, T cells are cultured without the stimulator tumor cells. In other experiments, T cells are restimulated with antigenically distinct cells. After six days, the cultures are tested for cytotoxicity in a 4 hour ⁵¹Cr-release assay. The spontaneous ⁵¹Cr-release of the targets should reach a level less than 20%. For the anti-MHC class I blocking activity, a tenfold concentrated supernatant of W6/32 hybridoma is added to the test at a final concentration of 12.5% (Heike M., et al., J. Immunotherapy 15:165-174).

4.15.3. Levels of Tumor Specific Antigens

Although it may not be possible to detect unique tumor antigens on all tumors, many tumors display antigens that distinguish them from normal cells. The monoclonal antibody reagents have permitted the isolation and biochemical characterization of the antigens and have been invaluable diagnostically for distinction of transformed from nontransformed cells and for definition of the cell lineage of transformed cells. The best-characterized human tumor-associated antigens are the oncofetal antigens. These antigens are expressed during embryogenesis, but are absent or very difficult to detect in normal adult-tissue. The prototype antigen is carcinoembryonic antigen (CEA), a glycoprotein found on fetal gut and human colon cancer cells, but not on normal adult colon cells. Since CEA is shed from colon carcinoma cells and found in the serum, it was originally thought that the presence of this antigen in the serum could be used to screen patients for colon cancer. However, patients with other tumors, such as pancreatic and breast cancer, also have elevated serum levels of CEA. Therefore, monitoring the fall and rise of CEA levels in cancer patients undergoing therapy has proven useful for predicting tumor progression and responses to treatment.

Several other oncofetal antigens have been useful for diagnosing and monitoring human tumors, e.g., alpha-fetoprotein, an alpha-globulin normally secreted by fetal liver and yolk sac cells, is found in the serum of patients with liver and germinal cell tumors and can be used as a marker of disease status.

4.15.4. Computed Tomographic (CT) Scan

CT remains the choice of techniques for the accurate staging of cancers. CT has proved more sensitive and specific than any other imaging techniques for the detection of metastases.

4.15.5. Measurement of Putative Biomarkers

The levels of a putative biomarker for risk of a specific cancer are measured to monitor the effect of hsp bound to peptide complexes. For example, in individuals at enhanced risk for prostate cancer, serum prostate-specific antigen (PSA) is measured by the procedure described by Brawer, M. K., et al., 1992, J. Urol. 147:841-845, and Catalona, W. J., et al., 1993, JAMA 270:948-958; or in individuals at risk for colorectal cancer, CEA is measured as described above in Section 4.5.3; and in individuals at enhanced risk for breast cancer, 16-α-hydroxylation of estradiol is measured by the procedure described by Schneider, J. et al., 1982, Proc. Natl. Acad. Sci. ISA 79:3047-3051.

4.15.6. Sonogram

A sonogram remains an alternative choice of technique for the accurate staging of cancers.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

What is claimed is:

1. A method of making a composition, which composition is immunogenic against a type of cancer or an agent of infectious disease, comprising mixing:
(a) an amount of a purified first complex comprising a first heat shock protein complexed to a first peptide which displays antigenicity of an antigen of said type of cancer or antigenicity of an antigen of an agent of said infectious disease; or an amount of a purified population of heterogeneous first complexes, said population of heterogeneous first complexes comprising a plurality of different first peptides; and
(b) an equal or greater amount of a second heat shock protein that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or cells infected with said agent of infectious disease, respectively.

2. The method according to claim 1, wherein the mass ratio of the first heat shock protein to the second heat shock protein is 1:1.

3. The method according to claim 1, wherein the mass ratio of the first heat shock protein to the second heat shock protein is 1:2.

4. The method according to claim 1, wherein the mass ratio of the first heat shock-protein to the second heat shock protein is 1:5.

5. The method according to claim 1, wherein the mass ratio of the first heat shock protein to the second heat shock protein is 1:10.

6. The method according to claim 1, wherein the mass ratio of the first heat shock protein to the second heat shock protein is 1:100.

7. The method according to claim 1, wherein the first complex is prepared from cancerous tissue of said type of cancer or a cell infected with said agent of infectious disease, respectively.

8. The method according to claim 1, wherein the first complex is prepared in vitro by complexing the first heat shock protein to a tumor specific antigen or an antigen of aid agent of said infectious disease, respectively.

9. The method according to claim 8, wherein the first heat shock protein or the tumor specific antigen or antigen of said agent of said infectious disease is recombinant.

10. The method according to claim 1, wherein the second heat shock protein is not complexed to any molecule.

11. The method according to claim 1, wherein the second heat shock protein is complexed to a second peptide to produce a second complex.

12. The method according to claim 11, wherein the second complex is produced in vitro.

13. The method according to claim 12, wherein the second complex is produced in a cultured cell.

14. The method according to claim 13, wherein the cultured cell recombinantly expresses the heat shock protein.

15. The method according to claim 1, wherein the second heat shock protein is present in a cell lysate or extract that is mixed with said amount of step (a).

16. The method according to claim 1, wherein the first heat shock protein and the second heat shock protein are the same.

17. The method according to claim 16, wherein the first heat shock protein and the second heat shock protein are each hsp70, hsp90, gp96, calreticulin, hsp 110, or grp170.

18. The method according to claim 1, wherein the first heat shock protein and the second heat shock protein are different.

19. The method according to claim 18, wherein the first heat shock protein is hsp70, hsp90, gp96, calreticulin, hsp 110, or grp170.

20. The method according to claim 18, wherein the second heat shock protein is hsp70, hsp90, gp96, calreticulin, hsp 110, or grp 170.

21. The method according to claim 1, wherein the first heat shock protein and first peptide are noncovalently linked to each other.

22. The method according to claim 21, wherein the second heat shock protein is noncovalently linked to a second peptide.

23. The method according to claim 21, wherein the second heat shock protein is covalently linked to a second peptide.

24. The method according to claim 21, wherein the second heat shock protein is in the form of a fusion protein comprising a second peptide.

25. The method according to claim 1, wherein the first heat shock protein and first peptide are covalently linked to each other.

26. The method according to claim 25, wherein the second heat shock protein is noncovalently linked to a second peptide.

27. The method according to claim 25, wherein the second heat shock protein is covalently linked to a second peptide.

28. The method according to claim 25, wherein the second heat shock protein is in the form of a fusion protein comprising a second peptide.

29. The method according to claim 1, wherein the first heat shock protein and first peptide are covalently linked to each other.

30. The method according to claim 16, wherein the second heat shock protein is noncovalently linked to a second peptide.

31. The method according to claim 16, wherein the second heat shock protein is covalently linked to a second peptide.

32. The method according to claim 16, wherein the second heat shock protein is in the form of a fusion protein comprising a second peptide.

33. The method according to claim 10, wherein the first complex is purified to apparent homogeneity, as detected on a SDS-PAGE gel.

34. The method according to claim 11, wherein the first complex is purified to apparent homogeneity, as detected on a SDS-PAGE gel.

35. The method according to claim 11, wherein the second complex is purified to apparent homogeneity, as detected on a SDS-PAGE gel.

36. The method according to claim 1, wherein the cancer is a sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenström's macroglobulinemia, and heavy chain disease.

37. The method according to claim 1 in which the infectious agent is a virus, bacterium, protozoa, fungus, or parasite.

38. A method of making a composition, which composition is immunogenic against a type of cancer or an agent of infectious disease, comprising:
(a) purifying a first heat shock protein-peptide complex from cancerous tissue of said type of cancer or metastasis thereof, or cells infected with said agent of infectious disease, respectively; and
(b) mixing an amount of said first complex with an equal or greater amount of a second heat shock protein that is not complexed in vitro to a peptide which displays antigenicity of an antigen of said type of cancer or antigenicity of an antigen of an agent of said infectious disease, respectively; and which second heat shock protein is not in the form of a complex, said complex having been isolated as a complex from cancerous tissue of said type of cancer or cells infected with said agent of infectious disease, respectively.

39. A method of making a composition, which composition is immunogenic against a type of cancer or an agent of infectious disease, comprising:
(a) complexing in vitro a purified first heat shock protein to a tumor-specific antigen of said type of cancer or an antigen of said agent of said infectious disease, respectively, to produce a first complex; and
(b) mixing an amount of said first complex with an equal or greater amount of a second heat shock protein that is not complexed in vitro to a peptide which displays antigenicity of an antigen of said type of cancer or antigenicity of an antigen of an agent of said infectious disease, respectively; and which second heat shock protein is not in the form of a complex, said complex having been isolated as a complex from cancerous tissue of said type of cancer or cells infected with said agent of infectious disease, respectively.

40. The method according to claim 38 or 39, wherein the second heat shock protein is not complexed to any molecule.

41. The method according to claim 38 or 39, wherein the second heat shock protein is complexed in vitro to a peptide which does not display antigenicity of an antigen of said type of cancer or antigenicity of an antigen of an agent of said infectious disease, respectively.

42. The method according to claim 38 or 39, wherein the second heat shock protein is present in a cell lysate or extract that is mixed with said amount of step (a).

43. A composition made by mixing:
(a) an amount of a purified first complex comprising a first heat shock protein complexed to a peptide which displays antigenicity of an antigen of said type of cancer or antigenicity of an antigen of an agent of said infectious disease; and
(b) an equal or greater amount of a second heat shock protein that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or cells infected with said agent of infectious disease, respectively.

44. A composition comprising:
(a) a purified first complex comprising a first heat shock protein complexed to a peptide which displays antigenicity of an antigen of a type of cancer or antigenicity of an antigen of an agent of an infectious disease; and
(b) an equal or greater amount of a second heat shock protein that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or the same tissue type, or cells infected with said agent of infectious disease or the same cell type, respectively;
wherein the composition is immunogenic against said type of cancer or said agent of infectious disease, respectively.

45. A method of eliciting an immune response against a type of cancer or against an agent of an infectious disease in an individual, comprising administering to the individual an amount of a composition effective to elicit an immune response against said type of cancer or said agent of infectious disease, said composition comprising:
(a) an amount of a purified first complex comprising a first heat shock protein complexed to a peptide which displays antigenicity of an antigen of said type of cancer or antigenicity of an antigen of an agent of said infectious disease; and
(b) an equal or greater amount of a second heat shock protein that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or the same tissue type, or cells infected with said agent of infectious disease or the same cell type, respectively.

46. The method according to claim 45, wherein the first heat shock protein is gp96, hsp70, hsp 110 or grp170, and the amount of the composition is in the range of 1 to 100 micrograms.

47. The method according to claim 46, wherein the amount of the composition is in the range of 2-50 micrograms.

48. The method according to claim 47, wherein the amount of the composition is in the range of 5-25 micrograms.

49. The method according to claim 45, wherein the first heat shock protein is hsp90, and the amount of the composition is in the range of 10-500 micrograms.

50. The method according to claim 49, wherein the amount of the composition is in the range of 20-400 micrograms.

51. The method according to claim 50, wherein the amount of the composition is in the range of 50-250 micrograms.

52. The method according to claim 45, wherein the first heat shock protein is calreticulin, and the amount of the composition is in the range of 0.5-50 micrograms.

53. The method according to claim 52, wherein the amount of the composition is in the range of 1-25 micrograms.

54. The method according to claim 53, wherein the amount of the composition is in the range of2.5-10 micrograms.

55. The method according to claim 45, wherein in which said administering step is repeated at weekly intervals.

56. The method according to claim 45, wherein said administering step is repeated five times, the first administration being on the left arm, the second administration being on the right arm, the third administration being on the left belly, the fourth administration being on the right belly, the fifth administration being on the left thigh, and the sixth administration being on the right thigh; said first through sixth administration being intradermally.

57. The method according to claim 45, wherein eliciting an immune response against a type of cancer is desired and the first complex is prepared from cancerous tissue of said type of cancer or a metastasis thereof autologous to the individual.

58. The method according to claim 45, wherein eliciting an immune response against a type of cancer is desired and the first complex is prepared from cancerous tissue of said type of cancer or a metastasis thereof allogeneic to the individual.

59. The method according to claim 45, further comprising administering to the individual an effective amount of a biological response modifier selected from the group consisting of interferon-α, interferon-γ, interleukin-2, interleukin-4, interleukin-6, and tumor necrosis factor.

60. The method according to claim 45, wherein the cancer is a sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenström's macroglobulinemia, and heavy chain disease.

61. The method according to claim 45 in which the infectious agent is a virus, bacterium, protozoa, fungus, or parasite.

62. A method of treating or preventing a type of cancer or an infectious disease in an individual in whom said treatment or prevention is desired, comprising administering to the individual a therapeutically effective amount of a composition, said composition comprising:
(a) an amount of a purified first complex comprising a first heat shock protein complexed to a peptide which displays antigenicity of an antigen of said type of cancer or antigenicity of an antigen of an agent of said infectious disease;
or an amount of a purified population of heterogeneous first complexes, said population of heterogeneous first complexes comprising a plurality of different first peptides; and
(b) an equal or greater amount of a second heat shock protein that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or the same tissue type, or cells infected with said agent of infectious disease or the same cell type, respectively.

63. The method according to claim 62, wherein the amount of the composition is in the range of 0.1 to 2 micrograms.

64. The method according to claim 62, wherein the amount of the composition is in the range of 5 to 20 micrograms.

65. The method according to claim 62, wherein the amount of the composition is in the range of 0.1 to 2 micrograms and the mass ratio of the first heat shock protein to the second heat shock protein is 1:10.

66. The method according to claim 65, wherein the first heat shock protein is hsp70 or gp96.

67. The method according to claim 62, wherein the amount of the composition is in the range of 5 to 20 micrograms and the mass ratio of the first heat shock protein to the second heat shock protein is 1:10.

68. The method according to claim 67, wherein first heat shock protein is hsp90.

69. The method according to claim 67, wherein the heat shock protein is hsp70or gp96.

70. The method according to claim 62, wherein in which said administering step is repeated at weekly intervals.

71. The method according to claim 62, wherein said administering step is repeated five times, the first administration being on the left arm, the second administration being on the right arm, the third administration being on the left belly, the fourth administration being on the right belly, the fifth administration being on the left thigh, and the sixth administration being on the right thigh; said first through sixth administration being intradermally.

72. The method according to claim 62, wherein the treatment or prevention of a type of cancer is desired and the first complex is prepared from cancerous tissue of said type of cancer or a metastasis thereof autologous to the individual.

73. The method according to claim 62, wherein the treatment or prevention of a type of cancer is desired and the first complex is prepared from cancerous tissue of said type of cancer or a metastasis thereof allogeneic to the individual.

74. The method according to claim 62, further comprising administering to the individual an effective amount of a biological response modifier selected from the group consisting of interferon-α, interferon-γ, interleukin-2, interleukin-4, interleukin-6, and tumor necrosis factor.

75. The method according to claim 62, wherein the cancer is a sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenström's macroglobulinemia, and heavy chain disease.

76. The method according to claim 62 in which the infectious agent is a virus, bacterium, protozoa, fungus, or parasite.

77. A method of making a composition, which composition is immunogenic against a type of cancer or an agent of infectious disease, comprising mixing:
(a) an amount of a purified first complex comprising a shock protein complexed to a first peptide which displays antigenicity of an antigen of said type of cancer or antigenicity of an antigen of an agent of said infectious disease; or an amount of a purified population of heterogeneous first complexes, said population of heterogeneous first complexes comprising a plurality of different first peptides; and
(b) an equal or greater amount of an α2M.

78. A method of making a composition, which composition is immunogenic against a type of cancer or an agent of infectious disease, comprising:
(a) purifying a heat shock protein-peptide complex from cancerous tissue of said type of cancer or metastasis thereof, or cells infected with said agent of infectious disease, respectively; and
(b) mixing an amount of said first complex with an equal or greater amount of an α2M.

79. A method of making a composition, which composition is immunogenic against a type of cancer or an agent of infectious disease, comprising:
(a) complexing in vitro a purified heat shock protein to a tumor-specific antigen of said type of cancer or an antigen of said agent of said infectious disease, respectively, to produce a first complex; and
(b) mixing an amount of said first complex with an equal or greater amount of an α2M.

80. A composition made by mixing:
(a) an amount of a purified first complex comprising a heat shock protein complexed to a peptide which displays antigenicity of an antigen of said type of cancer or antigenicity of an antigen of an agent of said infectious disease; and
(1b) an equal or greater amount of an α2M.

81. A composition comprising:
(a) a purified first complex comprising a heat shock protein complexed to a peptide which displays antigenicity of an antigen of a type of cancer or antigenicity of an antigen of an agent of an infectious disease; and
(b) an equal or greater amount of an α2M.

82. A method of eliciting an immune response against a type of cancer or against an agent of an infectious disease in an individual, comprising administering to the individual an amount of a composition effective to elicit an immune response against said type of cancer or said agent of infectious disease, said composition comprising:
(a) an amount of a purified first complex comprising a heat shock protein complexed to a peptide which displays antigenicity of an antigen of said type of cancer or antigenicity of an antigen of an agent of said infectious disease; and
(b) an equal or greater amount of an α2M.

83. A method of treating or preventing a type of cancer or an infectious disease in an individual in whom said treatment or prevention is desired, comprising administering to the individual a therapeutically effective amount of a composition, said composition comprising:
(a) an amount of a purified first complex comprising a heat shock protein complexed to a peptide which displays antigenicity of an antigen of said type of cancer or antigenicity of an antigen of an agent of said infectious disease;
or an amount of a purified population of heterogeneous first complexes, said population of heterogeneous first complexes comprising a plurality of different first peptides; and
(b) an equal or greater amount of an α2M.

84. A method of making a composition, which composition is immunogenic against a type of cancer or an agent of infectious disease, comprising mixing:
(a) an amount of a purified first complex comprising α2M complexed to a first peptide which displays antigenicity of an antigen of said type of cancer or antigenicity of an antigen of an agent of said infectious disease; and
(b) an equal or greater amount of a heat shock protein.

85. A method of making a composition, which composition is immunogenic against a type of cancer or an agent of infectious disease, comprising:
(a) purifying a α2M protein-peptide complex from cancerous tissue of said type of cancer or metastasis thereof, or cells infected with said agent of infectious disease, respectively; and
(b) mixing an amount of said first complex with an equal or greater amount of a heat shock protein that is not complexed in vitro-to a peptide which displays antigenicity of an antigen of said type of cancer or antigenicity of an antigen of an agent of said infectious disease, respectively; and which heat shock protein is not in the form of a complex, said complex having been isolated as a complex from cancerous tissue of said type of cancer or cells infected with said agent of infectious disease, respectively.

86. A method of making a composition, which composition is immunogenic against a type of cancer or an agent of infectious disease, comprising:
(a) complexing in vitro a purified α2M protein to a tumor-specific antigen of said type of cancer or an antigen of said agent of said infectious disease, respectively, to produce a first complex; and
(b) mixing an amount of said first complex with an equal or greater amount of a heat shock protein that is not complexed in vitro to a peptide which displays antigenicity of an antigen of said type of cancer or antigenicity of an antigen of an agent of said infectious disease, respectively; and which heat shock protein is not in the form of a complex, said complex having been isolated as a complex from cancerous tissue of said type of cancer or cells infected with said agent of infectious disease, respectively.

87. A composition made by mixing:
(a) an amount of a purified first complex comprising a α2M protein complexed to a peptide which displays antigenicity of an antigen of said type of cancer or antigenicity of an antigen of an agent of said infectious disease; and
(b) an equal or greater amount of a heat shock protein that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or cells infected with said agent of infectious disease, respectively.

88. A composition comprising:
(a) a purified first complex comprising a α2M protein complexed to a peptide which displays antigenicity of an antigen of a type of cancer or antigenicity of an antigen of an agent of an infectious disease; and
(b) an equal or greater amount of a heat shock protein that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or the same tissue type, or cells infected with said agent of infectious disease or the same cell type, respectively;
wherein the composition is immunogenic against said type of cancer or said agent of infectious disease, respectively.

89. A method of eliciting an immune response against a type of cancer or against an agent of an infectious disease in an individual, comprising administering to the individual an amount of a composition effective to elicit an immune response against said type of cancer or said agent of infectious disease, said composition comprising:
(a) an amount of a purified first complex comprising a α2M protein complexed to a peptide which displays antigenicity of an antigen of said type of cancer or antigenicity of an antigen of an agent of said infectious disease; and
(b) an equal or greater amount of a heat shock protein that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or the same tissue type, or cells infected with said agent of infectious disease or the same cell type, respectively.

90. A method of treating or preventing a type of cancer or an infectious disease in an individual in whom said treatment or prevention is desired, comprising administering to the individual a therapeutically effective amount of a composition, said composition comprising:
(a) an amount of a purified first complex comprising a α2M protein complexed to a peptide which displays antigenicity of an antigen of said type of cancer or antigenicity of an antigen of an agent of said infectious disease; or an amount of a purified population of heterogeneous first complexes, said population of heterogeneous first complexes comprising a plurality of different first peptides; and
(b) an equal or greater amount of a heat shock protein that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or the same tissue type, or cells infected with said agent of infectious disease or the same cell type, respectively.

91. A method of making a composition, which composition is immunogenic against a type of cancer or an agent of infectious disease, comprising mixing:
(a) an amount of a purified first complex comprising a first α2M complexed to a first peptide which displays antigenicity of an antigen of said type of cancer or antigenicity of an antigen of an agent of said infectious disease; or an amount of a purified population of heterogeneous first complexes, said population of heterogeneous first complexes comprising a plurality of different first peptides; and
(1,) an equal or greater amount of a second α2M that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or cells infected with said agent of infectious disease, respectively.

92. A method of making a composition, which composition is immunogenic against a type of cancer or an agent of infectious disease, comprising:
(a) purifying a first α2M-peptide complex from cancerous tissue of said type of cancer or metastasis thereof, or cells infected with said agent of infectious disease, respectively; and
(b) mixing an amount of said first complex with an equal or greater amount of a second α2M that is not complexed in vitro to a peptide which displays antigenicity of an antigen of said type of cancer or antigenicity of an antigen of an agent of said infectious disease, respectively; and which second α2M is not in the form of a complex, said complex, having been isolated as a complex from cancerous tissue of said type of cancer or cells infected with said agent of infectious disease, respectively.

93. A method of making a composition, which composition is immunogenic against a type of cancer or an agent of infectious disease, comprising:
(a) complexing in vitro a purified first α2M to a tumor-specific antigen of said type of cancer or an antigen of said agent of said infectious disease, respectively, to produce a first complex; and
(b) mixing an amount of said first complex with an equal or greater amount of a second α2M that is not complexed in vitro to a peptide which displays antigenicity of an antigen of said type of cancer or antigenicity of an antigen of an agent of said infectious disease, respectively; and which second α2M is not in the form of a complex, said complex having been isolated as a complex from cancerous tissue of said type of cancer or cells infected with said agent of infectious disease, respectively.

94. A composition made by mixing:
(a) an amount of a purified first complex comprising a first α2M complexed to a peptide which displays antigenicity of an antigen of said type of cancer or antigenicity of an antigen of an agent of said infectious disease; and
(b) an equal or greater amount of a second α2M that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or cells infected with said agent of infectious disease, respectively.

95. A composition comprising:
(a) a purified first complex comprising a first α2M complexed to a peptide which displays antigenicity of an antigen of a type of cancer or antigenicity of an antigen of an agent of an infectious disease; and
(b) an equal or greater amount of a second α2M that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or the same tissue type, or cells infected with said agent of infectious disease or the same cell type, respectively;
wherein the composition is immunogenic against said type of cancer or said agent of infectious disease, respectively.

96. A method of eliciting an immune response against a type of cancer or against an agent of an infectious disease in an individual, comprising administering to the individual an amount of a composition effective to elicit an immune response against said type of cancer or said agent of infectious disease, said composition comprising:
(a) an amount of a purified first complex comprising a first α2M complexed to a peptide which displays antigenicity of an antigen of said type of cancer or antigenicity of an antigen of an agent of said infectious disease; and
(b) an equal or greater amount of a second α2M that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or the same tissue type, or cells infected with said agent of infectious disease or the same cell type, respectively.

97. A method of treating or preventing a type of cancer or an infectious disease in an individual in whom said treatment or prevention is desired, comprising administering to the individual a therapeutically effective amount of a composition, said composition comprising:
(a) an amount of a purified first complex comprising a first α2M complexed to a peptide which displays antigenicity of an antigen of said type of cancer or antigenicity of an antigen of an agent of said infectious disease; or an amount of a purified population of heterogeneous first complexes, said population of heterogeneous first complexes comprising a plurality of different first peptides; and
(b) an equal or greater amount of a second α2M that is not complexed in vitro to a peptide which displays antigenicity of an antigen of said type of cancer or antigenicity of an antigen of an agent of said infectious disease, respectively; and is not in the form of a complex, said complex having been isolated as a complex from cancerous tissue of said type of cancer or the same tissue type, or cells infected with said agent of infectious disease or the same cell type, respectively.
